(19) United States
(12) Patent Application Publication
(10) Pub. No.: US 2023/0355884 A1
(43) Pub. Date: Nov. 9, 2023

(54) DELIVERY DEVICE

(71) Applicant: SULZER MIXPAC AG, Haag (Rheintal) (CH)

(72) Inventors: Herbert BECHTOLD, Denkingen (DE); Stefan GAUL, Stetten (CH); Robert LURF, Zürich (CH)

(21) Appl. No.: 17/777,524

(22) PCT Filed: Nov. 18, 2020
(86) PCT No.: PCT/EP2020/082582

§ 371 Date: May 17, 2022

Related U.S. Application Data
(60) Provisional application No. 63/013,811, filed on Apr. 22, 2020.
(60) Provisional application No. 62/937,014, filed on Nov. 18, 2019.

(51) Int. Cl. A61M 5/315

(52) U.S. Cl. CPC A61M 5/31551; A61M 5/31568; A61M 2205/3327

(57) ABSTRACT

A delivery device includes a housing, a controller for setting a dose and a sensor mechanism for recording the dose set by the controller. The controller rotates with respect to the housing around a longitudinal axis and moves axially with respect to the housing along the longitudinal axis during dose setting. The sensor mechanism includes an electronics module and a dose setting sensor with a first sensor element and a second sensor element. The electronics module and the first sensor element are rotationally and axially fixed with respect to the controller and the second sensor element is rotationally fixed with respect to the housing and rotationally movable with respect to the controller. The first and second sensor element are at least temporarily positioned outside of the housing along the longitudinal axis during dose setting.

### CROSS-REFERENCE TO RELATED APPLICATION

This application is a U.S. National Stage application of International Application No. PCT/EP2020/082582, filed Nov. 18, 2020, which claims priority to U.S. Provisional Application No. 62/937,014, filed Nov. 18, 2019 and U.S. Provisional Application No. 63/013,811, filed Apr. 22, 2020, the contents of each of which are hereby incorporated by reference.

### BACKGROUND

### Field of Invention

The present disclosure is directed at a delivery device, such as a drug delivery device, that has a sensor mechanism for recording a set dose and/or for sensing a travel of internal components which relates to the dose expelled by the delivery device.

In general, the present disclosure relates to the use of a sensor mechanism in medical devices to provide information relating to the status, changes, and operation of the medical device. Dose sensing mechanisms based on the use of ring-like structures, tubes, disk-like structures, or barrel shaped components and an end of dose sensor, used alone or in combination with a linear sensor, can be located on or within a medical injection device such that an electronics module can interrogate and receive signals from all or some of the sensors that reflects information directly related to the setting and/or delivery of a dose of drug or medicament. The electronics module can be an integral part of the sensor mechanism or can be a separate device that can be reused with a plurality of medical devices.

### Background Information

There are a vast number of conventional drug delivery devices on the market that are capable of performing any number of operations that benefit a user or patient. For example, there exist a variety of devices that automatically, semi-automatically or manually deliver one or more doses of medicament through injection (needle and needleless), inhalation, infusion, atomization, drops, patches, and implants. In each case it is important to monitor certain device attributes to support and guide patients during use of the device so as to increase therapy adherence. In particular, the knowledge of the dialed (set) and/or expelled (delivered) dose are important parameters for healthcare providers and users to know, monitor and record. Likewise, confirmation that the dose of medicament was actually performed and delivered completely (end of dose determination) is also a significant parameter to know and record.

For a number of users, medical devices for delivery of medicament, such as injection devices, can be overwhelming because of the mechanical sophistication of the devices. As a result, the detection of dialed or expelled dose is challenging. Furthermore, the inclusion of electronic sensors to automatically determine, transmit and/or record dosing parameters can make the availability of such devices cost prohibitive. As such, the additional cost for a sensor system must be kept as low as possible. Further, it is desirable that the integration of the additional sensing and connectivity solution should be designed and configured so as not lead to significant changes to the mechanical operation of the pen.

The ability to monitor set doses of medicament and the progress and delivery of set doses with one or more sensors would greatly benefit patient safety, drug administration or therapy compliance, device failure analysis, and future device design, just to name a few benefits. In some cases, medical devices are designed for non-medically trained individuals to selfadminister medicaments. For example, users of such devices include diabetics, where medication management and compliance, i.e. the degree to which a patient follows medical instructions and protocols, is often of extreme importance. To evaluate and determine compliance of a self-medicating user, it is desirable to obtain as much information about each injection as possible, for example, the determination of the actual dose of the medication injected, the amount of the set dose, whether a dose setting correction was needed, the rate of dose injection, whether the injection was halted, the time required to complete the injection, and confirmation that the dose was fully delivered. Collection and evaluation of such data can be especially important if the user is physical impaired, for example, having reduced eyesight or severe arthritis.

There are a handful of conventional medical devices that are constructed with drug delivery setting mechanisms that include one or more sensors that attempt to detect a variety of interacting mechanical components and functions, such as setting a dose, dose cancellation, and ultimately delivering the set dose. Each of the known devices has certain limitations, such as requiring the addition to the device of encoders that attempt to measure movement of several different parts of the device or that must contain dedicated switches that only capture limited data, such as activation of the device, but not the progress of dose delivery.

With the need to monitor, collect and evaluate medical device attributes, especially in drug delivery devices, it is desirable to provide medical devices, such as medication delivery systems, that are economical to manufacture and that can monitor, record, and report various device attributes and that can work wirelessly and with other devices. As such, it is an object of the present disclosure to provide medical devices that include one or more sensors to allow the above-mentioned device attributes to be monitored, measured, recorded and transmitted so that the collected data can be evaluated by patients, health care professionals, and device manufactures.

### SUMMARY

In general, the disclosure presented below achieves the above-mentioned goals, inter alia, by combining medical devices with stationary and rotating ring or barrel type sensors, or linear sensors, or end of dose switch or contact, that are used in combination with an electronic module.

The present disclosure thereby provides a delivery device. Embodiments are described in the description and the drawings.

According to a first aspect, the present disclosure is directed at a delivery device, such as a drug delivery or injection device, comprising:
a housing for receiving a cartridge for a drug, the housing having a longitudinal extent along a longitudinal axis,
a control element for setting a dose to be delivered by the delivery device and
a sensor mechanism for recording the dose set by the control element.

Thereby, the control element is configured to rotate with respect to the housing around the longitudinal axis during dose setting and to additionally axially move with respect to the housing along the longitudinal axis during dose setting. The sensor mechanism comprises an electronics module and a dose setting sensor for sensing the rotation of the control element during dose setting. The dose setting sensor comprises a first sensor element and a second sensor element, wherein the first sensor element has contact elements that are electrically connected to the electronics module via electrical conductors. The electronics module and the first sensor element are rotationally and axially fixed with respect to the control element and the second sensor element is rotationally fixed with respect to the housing and rotationally movable with respect to the control element. Furthermore, the first sensor element and the second sensor element are at least temporarily positioned outside of the housing along the longitudinal axis during dose setting.

Mounting the electronics module and the first sensor element fixedly to the control element enables a compact sensor mechanism that can be, for example, completely integrated into the control element. Such integration is further assisted by the first and second sensor element being at least temporarily located outside of the housing during dose setting. The housing of the delivery device can comprise all members of the delivery device that do not axially move with respect to a cartridge holder for receiving the drug container. The incorporation of such a sensor mechanism into existing device designs only adds a minimum manufacturing cost to the already existing devices. Furthermore, the electronics module is located at an easily accessible position which ensures good usability of the delivery device.

The control element can be located at a distal end of the delivery device. It can be configured as a rotary knob. Such a knob can also be termed dose knob or dose setting knob. Alternatively, it can also be configured, for example, as a rotary cylinder or sleeve that surrounds the delivery device, for example at its distal end.

With some embodiments, the first and second sensor element can be located permanently outside of the housing during dose setting. It also can be permanently located outside of the housing during dose delivery, for example at the end of the delivery of a set dose. With other embodiments, the first and second sensor element can also be temporarily located within the housing in the axial direction during dose setting and/or dose delivery, for example at the end of the delivery of the set dose or upon a setting of small doses. For example, the first and second sensor element can be configured to move out of the housing during dose setting.

The first and second sensor element can be connected to the housing via a connection member. The connection member can also connect the control element with the housing. The connection member can be configured to axially move with respect to the housing during dose setting. The connection member can, for example, be rotationally fixed with respect to the housing during dose setting.

According to an embodiment, the second sensor element is axially fixed with respect to the control element. This enables a robust and compact dose setting sensor.

According to an embodiment, the control element is connected to the housing via a connection member, wherein the connection member is axially movable and rotationally fixed with respect to the housing during both dose setting and dose delivery. The control element is rotationally movable with respect to the connection member during dose setting and the second sensor element is rotationally fixed with respect to the connection member. Such a connection member provides a reference member for sensing a relative rotation of the control element with respect to the housing even if the dose delivery sensor has been moved out of the housing during dose setting.

The movement of the connection member can be proportional to the dose set via the control element. Delivery of the set dose can then be achieved by pushing the control element and the connection member back into the housing. To this end, the delivery device can comprise a dosing mechanism that translates the linear axial movement of the connection member into axial movement of a piston rod that expels the drug to be delivered out of the delivery device. The connection member can be configured, for example, as a sleeve, such as a longitudinal sleeve that surrounds the dosing mechanism of the delivery device. The sleeve can be a dose dial sleeve. It also can be termed injection sleeve as it is the case with U.S. Pat. No. 8,512,296 B2.

According to an embodiment, the control element is axially movable with respect to the connection member from a dose setting position into a dose delivery position, wherein, for example, the control element and the first sensor element are rotationally fixed with respect to the connection member in the dose delivery position. Axial movement of the control element into the dose delivery position can switch the dosing mechanism from a dose setting state into a dose delivery state.

The dosing mechanism can comprise, for example, a metering element that is axially fixed and rotationally movable with respect to the housing. The metering element can be connected to the piston rod via a first threaded connection and to the connection member via a second threaded connection. In the dose setting state of the dosing mechanism, the piston rod can be rotationally fixed with respect to the metering element and rotationally movable with respect to the housing so that the piston rod rotates in unison with the metering element and does not axially move with respect to the housing. In the dose delivery state of the dosing mechanism, the piston rod can be axially movable with respect to the metering element and rotationally fixed with respect to the housing so that rotation of the metering element urges the piston rod into the proximal direction via the first threaded connection. Rotation of the metering element during dose setting can, for example, be induced via the second threaded connection by pushing the connection member into the proximal direction.

A delivery device having such a dosing mechanism is described, for example, in publication U.S. Pat. No. 8,512,296 B2 and the dosing mechanism described in this publication is incorporated into the present disclosure by reference. In U.S. Pat. No. 8,512,296 B2, the connection member is termed “injection sleeve”.

According to an embodiment, the second sensor element is axially movable with respect to the connection member. This allows the second sensor element being axially fixed with respect to the control element even if the control element is axially movable with respect to the connection member.

According to an embodiment, the second sensor element is connected to the connection member via a keyed connection, the keyed connection comprising, for example, at least one lug that is slideably received within a longitudinal recess orientated parallel to the longitudinal axis. Such a keyed connection allows for an easy yet sturdy rotational fixation of the second sensor element with respect to the connection member and the housing while allowing the second sensor element to move in the axial direction. The keyed connection can be configured directly between the second sensor element and the connection member or it can be configured, for example, between the second sensor element and a further member of the delivery device that is rotationally fixed with respect to the connection member.

According to an embodiment, the contact elements of the first sensor element are configured as two-dimensional surface contacts. This results in a mechanically simple first sensor element and allows for easy connection between the contact elements and the conductors that electrically connect the first sensor element with the electronics module.

According to an embodiment, the contact elements of the first sensor element are arranged on a cylindrical surface that is orientated parallel to the longitudinal axis. Such contact elements are easily accessible for being contacted by the second sensor element. The cylindrical surface can be a lateral area of a cylindrical carrier that is rigidly connected to the control element. Thereby, the cylindrical surface can be an inner or outer lateral area of the cylindrical carrier.

According to an embodiment, the second sensor element is configured to electrically contact the contact elements of the first sensor element in radial directions perpendicular to the longitudinal axis. This allows for firm electrically contact between the first and second sensor element.

According to an embodiment, the dose setting sensor comprises an insulating carrier that supports the electrical conductors and the contact elements of the first sensor element. Placing the electrical conductors and the contact elements of the first sensor element on the same insulating carrier allows for easy and cost-efficient manufacture of the sensor mechanism.

According to an embodiment, the insulating carrier is configured as a rigid and/or free-standing structure, wherein the electrical conductors and/or the contact elements of the first sensor element are rigidly attached to the carrier, for example as conductive inserts and/or co-molded with the insulating carrier. This yields a compact and sturdy sensor mechanism.

According to an embodiment, the insulating carrier is a printed circuit board, such as a flexible printed circuit board. Such printed circuit boards are easily manufacturable and allow for a precise placement of the electrical conductors and/or the contact elements.

According to an embodiment, the insulating carrier has a ring-shaped section supporting the first sensor element and a longitudinal section supporting the electrical conductors, wherein the ring-shaped section circumferentially extends around the longitudinal axis and the longitudinal section longitudinally extends in parallel to the longitudinal axis. The ring-shaped section can, for example, be formed by a flexible insulating carrier, such as a flexible printed circuit board, that is bent around the longitudinal axis.

According to an embodiment, the second sensor element is configured as a conductive metal element with an integrally formed linking structure contacting the first sensor element. This allows for cost efficient manufacture of a compact and sturdy second sensor element. The linking structure can comprise several metallic linking elements that are configured as freestanding and/or spring-loaded elements that are configured to bear against the contact elements of the first sensor element, for example in a radial direction, such as an inward or outward radial direction.

According to an embodiment, the second sensor element is configured as a punched and bent sheet metal.

According to an embodiment, the second sensor element comprises a conductive metal ring holding a plurality of linking elements of the linking structure, wherein the linking elements are configured to electrically connect at least two contact structures of the first sensor element with each other. For example, both the metal ring and the linking elements of the linking structure can be manufactured from a single piece of metal, such as a single sheet metal.

According to an embodiment, the dose setting sensor comprises a rotary dose setting encoder that generates sensor signals having electrical pulses upon rotation of the control element during dose setting and the electronics module is configured to determine the set dose from a number of the electrical pulses generated by the dose setting sensor. This allows for easy detection of incremental rotations of the control element.

According to an embodiment, the dose setting sensor is configured to generate the electrical pulses of the sensor signals at a rate that is proportional to the angular velocity of rotation of the control element.

According to an embodiment, the dose setting sensor is configured to provide a sensor signal to the electronics module that is indicative of a direction of rotation of the control element. This allows the electronics module to distinguish between rotations of the control element in one rotational direction that cause dose setting, i.e. an increase of the set dose, and rotations of the control element in the opposite direction that cause dose cancellation, i.e. a decrease of the set dose. The sensor signal can be a combined signal that comprises several, for example two, signals as signal components. For example, the sensor signal can comprise two signals as signal components whereby the signal components are generated in quadrature.

According to an embodiment, the electrical conductors comprise a first conductor and a second conductor and the first sensor element comprises a first contact structure conductively connected to the first conductor and a second contact structure conductively connected to the second conductor. Thereby, the second sensor element comprises a/the linking structure that is configured to repeatedly open and close an electrical contact between the first and second contact structure upon rotation of the control element. This can generate a pulsed electrical signal upon rotation of the control element that is easily detectable by the electronics module.

According to an embodiment, the linking structure comprises a first linking element and a second linking element conductively connected to the first linking element, wherein the second linking element is configured to sequentially move into electrical contact with the individual contact elements of the second contact structure upon rotation of the control element while the first linking element is in electrical contact with the first contact structure and to thereby sequentially connect the individual contact elements of the second contact structure with the first contact structure.

According to an embodiment, the first contact structure comprises a single one of the contact elements and the first linking element is configured to conductively contact the single contact element of the first contact structure while the second linking element sequentially moves into the electrical contact with the contact elements of the second contact structure. This allows for easy generation of a pulsed electrical signal.

According to an embodiment, the first contact structure and the second contact structure are circumferentially arranged after each other around the longitudinal axis in a way that, while a rotational position of the control element is within a first angular range, the first linking element contacts the first contact structure and the second linking element contacts the second contact structure, and that, while the rotational position of the control element is within a second angular range, the first linking element contacts the second contact structure, and that, while the rotational position of the control element is within a third angular range, the second linking element contacts the first contact structure. Thereby, the second angular range can be different from the third angular range.

With such a configuration, the individual contact elements of the first, second and third contact structure can be placed next to each other along a circumferential direction around the longitudinal axis. This allows for a compact sensor mechanism that occupies little installation space.

According to an embodiment, the electrical conductors comprise a third conductor and the first sensor element comprises a third contact structure conductively connected to the third conductor. Thereby, the linking structure of the second sensor element is configured to repeatedly open and close a further electrical contact between the first and third contact structure upon rotation of the control element. By establishing both the electrical contact of the second contact structure and the further electrical contact of the third contact structure with the single first contact structure, two separate and independent signals can be generated with a compact arrangement of the first and second sensor elements.

According to an embodiment, contact elements of the second contact structure and contact elements of the third contact structure are offset with respect to each other so that, upon rotation of the control element, the opening and closing of the electrical contact between the first and second contact structure exhibits a temporal shift with respect to the opening and closing of the further electrical contact between the first and third contact structure. The electronics module is then configured to determine a direction of rotation of the control element from the temporal shift. Thus, a rotation of the control element leading to dose setting can be discerned from a rotation leading to dose cancellation. The offset between the individual contact elements can be configured so that a first sensor signal generated by repeatedly closing an electrical contact between the first and second contact structure and a second sensor signal generated by repeatedly closing of an electrical contact between the first and third contact structure are generated in quadrature.

According to an embodiment, the control element is configured to axially move with respect to the housing during dose delivery, for example in a proximal direction, and the sensor mechanism comprises a dose delivery sensor that is configured to sense a delivery of the set dose by detecting the axial movement of the control element. When sensing both the dose set by the rotation of the control element and the dose actually delivered by the delivery device, the electronics module can check whether a set dose has been completely delivered.

Furthermore, the electronics module can be configured to alert the user of the delivery device of an incomplete dose delivery. By detecting the axial movement of the control element during dose delivery, the delivered dose can be detected also with devices, in which the control element is rotationally fixed during dose delivery so that the dose setting sensor does not generate any sensor signals.

The dose delivery sensor can be configured as a linear sensor, such as an incremental linear sensor. The dose delivery sensor can be configured to sense relative axial movement between two members of the delivery device that axially move with respect to each other when the control element axially moves during dose delivery. For example, the dose delivery sensor can be configured to sense axial movement between the housing and the connection member connecting the control element with the housing.

According to an embodiment, the dose delivery sensor comprises a sensor portion that is rotationally fixed with respect to the housing and an electrical connector for conductively connecting the rotationally fixed sensor portion to the electronics module. The electrical connector is configured to be in an open state during dose setting and to be transferred into a closed state during delivery of the set dose, for example at a beginning of the delivery of the set dose. Having a sensor portion that is rotationally fixed with respect to the housing allows for easy detection of relative movement between the housing and the control element. Furthermore, the open state of the electrical connector during dose setting disconnects the rotationally fixed portion from the rotating electronics module and control element during dose setting. This simplifies the electrical connection between the electronics module and the rotationally fixed sensor portion of the dose delivery sensor, as the necessity for providing sliding contacts is dispensed with.

According to an embodiment, the electrical connector is in the open state when the control element is in the dose setting position and the electrical connector is transferred into the closed state when the control element is moved into the dose delivery position. The closing of the electrical connector is therefore coupled to the movement of the control element into the dose delivery position, so that this movement can be detected by the closing of the electrical connector. For example, the electronics module can be configured to sense the closing of the electrical connector and to thereby detect the movement of the control element into the dose delivery position.

According to an embodiment, the electrical connector comprises a first part that is rotationally fixed with respect to the control element and the electronics module and that is conductively connected to the electronics module, and a second part that is rotationally fixed with respect to the housing. The first part is thereby axially and rotationally movable with respect to the second part. The first part can be, for example, permanently connected to the electronics module during operation of the delivery device.

According to an embodiment, the electrical connector comprises a circumferential contact arrangement and a connector contact. The circumferential contact arrangement is thereby circumferentially arranged about the longitudinal axis and rotationally and axially movable with respect to the connector contact. The electrical connector is configured to be transferred into the closed state by axial movement of the circumferential contact arrangement with respect to the connector contact and the connector contact is configured to electrically contact the circumferential contact arrangement in a closed state of the electrical connector. A circumferential contact arrangement enables closing of the electrical contact at every settable rotational position of the control element.

According to an embodiment, individual contacts of the circumferential contact arrangement are circumferentially distributed around the longitudinal axis and the electrical connector is only transferrable into the closed state if the circumferential contact arrangement is positioned at distinct and separated rotational positions with respect to the connector contact. The connector contact then contacts different sets, for example different pairs, of the contacts of the circumferential contact arrangement when being positioned at the individual rotational positions. This allows for reliable contact between the circumferential contact arrangement and the connector contact.

According to an embodiment, the first part comprises the circumferential contact arrangement and the second part comprises the connector contact. This allows for a compact configuration of the second part that only occupies installation space within a limited circumferential section of the delivery device.

According to an embodiment, the dose delivery sensor is configured as an end of dose switch that is configured to be actuated, for example to be closed, upon full delivery of the set dose. This allows for simple detection of the completion of the delivery of the set dose.

According to an embodiment, the end of dose switch is actuated by transferring a further electrical connector from an open state into a closed state, wherein the rotationally fixed sensor portion comprises the further electrical connector and the electrical connector is configured to be transferred from the open state into the closed state upon beginning of dose delivery. Thereby, the operation of the end of dose switch is independent from the operation of the electrical connector for connecting the dose delivery sensor to the electronics module. This allows for reliable detection of the closing of the end of dose switch by the electronics module. Furthermore, the electronics module can additionally detect from the closing of the electrical connector the beginning of the delivery of the set dose. This allows, for example, the electronics module to determine the time needed for delivering the set dose by measuring a time interval in between closing of the electrical contact and closing of the further electrical contact.

According to an embodiment, the dose delivery sensor is configured as a linear sensor that is configured to sense an axial movement of the control element along the longitudinal axis during dose delivery. The electronics module can therefore monitor the complete process of dose delivery. For example, the electronics module can be configured to detect interruptions or delays during dose delivery from a change of the axial movement of the control element. This enhances the information available for monitoring the use of the dose delivery device.

According to an embodiment, the rotationally fixed sensor portion comprises a dose delivery encoder that is elongated along the longitudinal axis and that is axially movable with respect to the housing. A rotationally fixed longitudinal encoder allows for a simple construction of the dose delivery sensor.

According to an embodiment, the dose delivery encoder is configured to repeatedly contact a static electrical contact upon axial movement, wherein the static electrical contact is axially fixed with respect to the housing. The electronics module is then configured to monitor the repeated contacting events between the dose delivery encoder and the static electrical contact and to determine the axial movement of the control element from the repeated contacting events. Such a linear incremental encoder can be easily integrated into existing dose delivery devices.

According to an embodiment, the electronics module is configured to monitor the repeated contacting events by counting a number of the repeated contacting events and to determine the axial movement of the control element from the counted number of repeated contacting events.

According to an embodiment, the electronics module is configured to determine a set dose from the rotation of the control element sensed by the dose setting sensor and to determine an injected dose from the axial movement sensed by the dose delivery sensor, for example upon a stopping of the axial movement of the control element and/or upon a release of the control element. The electronics module is further configured to compare the injected dose with the set dose, for example for detecting an only partial delivery of the set dose. This enhances the usability of the delivery device. For example, the electronics module can be configured to alert a user of the delivery device about the only partial delivery of the set dose. Furthermore, the electronics module can be configured to transmit information of only partially delivered doses for evaluation, for example via a wireless transmitter integrated into the electronics module.

In general, the aspect of a combination of a rotationally fixed dose delivery sensor with a rotationally movable electronics module via the connector contact is independent from the sensor mechanism also comprising a dose setting sensor. Furthermore, that combination is also independent from the exact mechanical configuration of the dosing mechanism of the delivery device.

According to a second aspect, the present disclosure is therefore directed at a delivery device comprising:
a housing for receiving a container for a drug, the housing having a longitudinal extent along a longitudinal axis,
a control element for setting a dose to be delivered by the delivery device and
a sensor mechanism for recording the dose delivered by the delivery device.

The control element is thereby configured to rotate with respect to the housing around the longitudinal axis during dose setting. The sensor mechanism comprises a dose delivery sensor that is configured to sense delivery of the set dose and an electronics module for evaluating sensor signals provided by the dose delivery sensor, wherein the electronics module is rotationally fixed with respect to the control element. The dose delivery sensor comprises a sensor portion that is rotationally fixed with respect to the housing and an electrical connector for conductively connecting the rotationally fixed sensor portion to the electronics module. The electrical connector is thereby configured to be in an open state during dose setting and to be transferred into a closed state during delivery of the set dose, for example at a beginning of the delivery of the set dose.

Embodiments of the delivery device according to the second aspect of the present disclosure can have one, several or all of the features that have been described in the preceding sections for the delivery device according to the first aspect of the present disclosure.

According to a third aspect, the present disclosure is also directed at a delivery device comprising:
a housing for receiving a container for a drug, the housing having a longitudinal extent along a longitudinal axis,
a control element for setting a dose to be delivered by the delivery device and
a sensor mechanism for recording the dose set by the control element and for recording the dose delivered by the delivery device.

The control element is thereby configured to rotate with respect to the housing around the longitudinal axis during dose setting. The sensor mechanism comprises a dose setting sensor that is configured for sensing the rotation of the control element during dose setting, a dose delivery sensor that is configured to sense delivery of the set dose, and an electronics module for evaluating sensor signals provided by the dose setting sensor and the dose delivery sensor. Thereby, the dose setting sensor is configured as a rotational sensor and the dose delivery sensor is configured as a linear sensor.

Embodiments of the delivery device according to the third aspect of the present disclosure can have one, several or all of the features that have been described in the preceding sections for the delivery device according to the first aspect of the present disclosure.

The present disclosure is also directed to sensor mechanisms and to number of medical devices that can incorporate such mechanisms, including, but not limited to, devices that automatically, semi-automatically or manually deliver one or more doses of medicament through injection (needle and needleless), inhalation, infusion, atomization, drops, patches, and implants. Incorporating one or more rotating or linear sensors into these medical devices that can communicate with an electronics module, enables the determination of a set dose, correction of a set dose, the beginning of dose delivery, the progress of dose delivery, and the end of dose delivery. Some or all of these parameters can be electronically monitored, measured, recorded, and transmitted remotely. The electronics module of the sensor mechanism of the present disclosure can be incorporated into the medical device, or be removably attached to the medical device, or be a completely stand-alone component.

In one non-limiting embodiment of the present disclosure there is a sensor mechanism for recoding a set dose in a medical device, such as an injection device, that includes a first sensor ring rotatably fixed relative to a housing of an injection device and a second sensor ring rotatable relative to the first sensor and the housing, where either the first or the second sensor ring comprises a plurality of contact surfaces, additionally these contact surfaces could be distributed on further rings or similar shaped structures. Although the present disclosure uses the term “ring” to describe some of the sensors that can be used in the sensor mechanism, the term “ring” should not be construed as limiting the exact shape of sensor. For example, the ring sensors described herein would also include sensors shaped like a barrel, tube or a cylinder. Indeed, a “ring” can be thought of as a transverse slice or segment of a hollow cylinder or barrel. In those embodiments where ring-like sensors are used, the contacts are aligned longitudinally in the device. This contrasts with those embodiments where the sensors are barrel shaped (tube or cylinder) which will have radial aligned contacts.

Electrical leads are attached to a contact point located on either the first or second sensor ring and an electronics module containing a microcontroller is electrically connected to the electrical leads. A battery can be connected to the microcontroller to supply power to the sensor mechanism. Relative rotation between the first and second sensor rings during a setting of a dose of medicament causes the contact point to move into and out of electrical contact with the plurality of contact surfaces such that the contact point can only be in electrical contact with one of the plurality of contact surfaces at a single point in time.

The above described sensor mechanism can further include a ring aligner positioned within the housing of an injection device, where the ring aligner can rotate relative to the housing during the dose setting. In a preferred design, the ring aligner can be rotatably fixed to the dose knob such both rings rotate in unison together. The plurality of contact surfaces can be located on the first sensor ring and the contact point can then be located on the second sensor ring which is rotationally fixed to the ring aligner such that the second sensor ring is rotationally fixed relative to the housing. In this configuration the leads all rotate relative to the first sensor ring during dose setting. It is preferable in some configurations of the sensor mechanism of the present disclosure to have the plurality of contact surfaces be configured so that they have an identical shape and dimension and to have each of the plurality of contact surfaces be separated from an adjacent contact surface by an identical distance, where that distance contains a nonelectrical conductive surface.

In some designs of the sensor mechanism the ring aligner can be non-rotatable relative to the housing during delivery of the set dose of medicament and the plurality of contact surfaces can be arranged circumferentially around an outside surface of the first sensor ring.

The microcontroller and the battery (part of the electronics module) in the sensor mechanism can both rotate with the ring aligner. The microcontroller can also contain a wireless communication module. A third sensor ring can be rotatably fixed relative to the housing and can also contain a plurality of contact surfaces. Additionally, the sensor mechanism could include a linear sensor located on a sleeve that is configured to move axially in a proximal direction during delivery of the set dose, where the linear sensor is electrically connected to the microcontroller during delivery of the set dose. This linear sensor could be configured to contact or engaged a conductive strip that is axially fixed to the housing such that during delivery of set dose of medicament, the linear sensor moves relative to and contacts the conductive strip. The microcontroller could monitor the relative movement of the linear sensor relative to the conductive strip and could determine an amount of medicament actually delivered compared to the set dose of medicament.

In yet another embodiment of the present disclosure there is presented a sensor mechanism for sensing the setting of dose, the cancellation of a set dose, a dose adjustment and the final set dose of medicament in an injection device where a ring aligner is positioned within an injection device housing such that it can rotate relative to the housing during the setting of a dose of medicament. The sensor mechanism includes a first sensor ring rotatably fixed to the housing, where the first sensor ring comprises a plurality of contact surfaces. A second sensor ring is rotatably fixed relative to the ring aligner and a third sensor ring is also included having a plurality of contact surfaces. This third sensor ring is rotatably fixed relative to the first sensor. Electrical leads are attached to the second sensor ring at a first contact point located on a first side surface and at a second contact point located on a second side surface. Further, the first and second contact points are not in alignment. A microcontroller is also included and is electrically connected to the electrical leads and a battery is connected to and supplies the power needed by the microcontroller.

In the just described embodiment, the rotation of the second sensor ring relative to the first and the second sensor rings causes the first and second contact points to move into and out of electrical contact with the plurality of contact surfaces such that the microcontroller can determine a first direction or a second direction of rotation of the ring aligner. The non-alignment of the first contact point with the second contact point can be configured such that the first contact point and the second contact point can both be in electrical contact with the plurality of contact surfaces at the same point in time.

This sensor mechanism can be designed such that the first direction of rotation occurs during dose setting and the second direction of rotation occurs during dose cancellation or adjustment. Additionally, a linear sensor can be positioned on a sleeve component of the medical device that moves axially in a proximal direction during delivery of the set dose of medicament and can be electrically connected to the microcontroller during delivery of the set dose. The injection device can also include a conductive strip fixed to the housing such the linear sensor moves relative to and contacts the conductive strip during delivery of the set dose. When this occurs, the microcontroller monitors the relative movement of the linear sensor relative to the conductive strip. This monitoring will allow the microcontroller to determine the start of the dose delivery, the time of delivery, whether there is an interruption in the delivery, the end of the delivery and the amount of medicament actually delivered compared to the set dose of medicament that can be determined from the one or more ring sensors.

Yet another embodiment of the sensor mechanism disclosed in this disclosure also senses a set dose in an injection device. Here a ring aligner is positioned within the housing of the injection device such that the ring aligner can rotate relative to the housing during a setting of a dose of medicament. A first sensor ring is rotatably fixed to the housing and contains a plurality of contact surfaces. A second sensor ring rotatably fixed relative to the ring aligner and a third sensor ring, also having a plurality of contact surfaces, is rotatably fixed relative to the first sensor. Electrical leads are attached to the second sensor ring at a first contact point located on a first side surface and are also attached at a second contact point located on a second side surface, where the first and second contact points are not in alignment. The sensor mechanism further includes a linear sensor located on a sleeve of the injection device that is configured to move axially in a proximal direction during delivery of the set dose of medicament.

The just described sensor mechanism can be extended by a conductive strip fixed to the housing of the injection device such that the linear sensor moves relative to and contacts the conductive strip during delivery of the set dose. As with the other described embodiments, a microcontroller is electrically connected to the electrical leads and to the linear sensor only during delivery of the set dose of medicament. A battery is electrically connected to the microcontroller. The rotation of the second sensor ring relative to the first and the second sensor rings causes the first and second contact points to move into and out of electrical contact with the plurality of contact surfaces such that the microcontroller can determine a first direction or a second direction of rotation of the ring aligner. The first direction of rotation is indicative of dose setting and the second direction of rotation is indicative of dose cancellation or dose adjustment from an inadvertently set dose to a lower dose.

The sensor mechanism can also have a switch that is activated only during delivery of the set dose, where the activation of the switch connects the microcontroller to the linear sensor. The switch can contain contact pins, where during switch activation the contact pins make electrical contact with the linear sensor to establish an electrical communication between the microcontroller, the conductive strip and the linear sensor. The microcontroller then monitors the axial movement of the linear sensor relative to the fixed conductive strip on the housing after the switch is activated and can determine the parameters mentioned above, especially the amount of medicament actually delivered compared to the set dose of medicament.

In addition, the sensor mechanism can include an end of dose delivery sensor that is configured as a mechanical switch or physical interaction of electrical contacts (collectively referred to below as an “EOD Switch”). The status of the EOD switch is monitored by the microcontroller and that status data can be transmitted via a wireless interface to an external device (e.g., smart phone, tablet, docking station, etc.). The EOD Switch status can also be transmitted to mobile network. One preferred location of the EOD Switch is in a fixed position within the dose setting mechanism and located proximally from a linearly moving dose dial sleeve. The switch is placed in such that the dose dial sleeve will contact the EOD Switch at exactly the same time when the dose dial sleeve reaches and is at zero dose position. During the setting of a dose, the dose dial sleeve moves linearly relative to the device housing in the distal direction away from the EOD Switch and simultaneously causes the switch to move to an open position, i.e., opening the electrical circuit. In some drug delivery device designs the dose dial sleeve moves both rotationally and linearly during dose setting and dose delivery.

During the process of delivering the set dose of medicament, the dose dial sleeve is moved linearly relative to the device housing until the EOD Switch closes which coincides with the complete delivery of the set dose of medicament and only when the dose dial sleeve is at zero position. If the drug is not completely delivered the EOD Switch is not closed and that information is communicated to the external device and ultimately to the user of the device. Once the dose is completely injected the EOD Switch will close and the status of the switch is read by the microcontroller and the dose can be calculated, along with the time of dose delivery, and this data can be transmitted via a wireless interface to an external device or directly into a mobile network.

When the medical device is configured as an injection device, there is included a medicament container, a medicament delivery mechanism operatively associated with the medicament container, and at least one of a number of different possible sensor mechanisms, including those specifically described above. The microcontroller and battery are part of an electronics module that can be integral with the injection device or can be contained in a separate housing that is operatively associated with sensor mechanism, for example, the electronics module can be removably attached to the medical device and reusable, and it also can have a user interface to allow a user to input instructions or commands through a screen or buttons. The electronics module is configured to communicate the ring and linear sensors as part of the sensor mechanism and to receive one or more signals from the sensors, where the received signals are processed and transformed to generate reportable information about the sensor mechanism itself that includes the position and motion of select mechanical components (linear and rotational). The electronics module can also be configured to communicate with a remote device having a user interface.

The electronics module would continuously monitor the sensors and would receive signal(s) indicative of dose setting, dose cancellation, dose adjustments, the start of dose delivery, dose delivery interruption, dose delivery progress and dose delivery completion. The microcontroller or microprocessor in the electronics module would process and transform those signals into reportable information concerning these injection device parameters. It is to be noted that signals relating to one or more of the parameters mentioned can be captured only when the electronics module is activated or in an energized sending/receiving state while the medical device is being used, such as during dose setting or dose delivery operations. This information could then be reported in real time to a display that is part of the electronics module or is part of another reporting device, such as a dedicated remote device, a cell phone or a desktop computer. Alternatively, the electronics module could record and store the information until the information was transmitted to another device automatically or be manually interrogated by a user using a separate remote device.

One possible electronics module of the present invention includes a microcontroller having a signal processing unit that can operate using a decoding algorithm having the capability to decode the signals obtained from the first, second or third sensors. These decoded signals can be stored within a memory associated with the electronics module. Those skilled in the art will appreciate that a wide variety of corresponding algorithms tables can be provided. As mentioned, the electronics module comprises a power supply and can include an application-specific integrated circuit (ASIC) to generate and receive signals to and from the one or more sensors described above that are part of the sensor mechanism. The ASIC can be adapted to collect information regarding the operation of the injection device and to transform the information collected into a format recognizable to a user or healthcare provider. A processor within the electronics module could use the received signals from the interrogated sensors to calculate or determine, for example, a set dose of medicament or an actual dose delivered. This calculated result could be transmitted to a display accessible by the user and it could be stored in a memory for transmission to another device via wired or wireless connection. The processor could also include a clock function to allow it to monitor the time/date of injection, the speed of injection, and whether the injection was halted and for how long. It is also possible that the electronics module could determine the temperature of device and hence provide an approximate temperature of the medicament at the time of injection or during non-use of the device. Temperature profiles of the injection device can be related to the effectiveness of medicaments, e.g., degree of degradation or reduced potency of the medicament.

As mentioned, the information obtained from the sensor mechanisms described above can be obtained through electrical leads that are connected to the one or more sensors and also to the electronics module such that information can be sent to a portable or remote device that can be dedicated to the electronics module. The portable device can be a smartphone or tablet or other portable device such as a laptop or a handheld personal data assistant (PDA), such as a personal diabetes manager (PDM), which has a processing device, memory, display, user interface and communications interface.

The portable device can be used wirelessly to program the electronics module with customized medicament delivery instructions, data logging and data integration procedures to provide a user with convenient access to accurate information on the electronics module and/or the portable device display or screen to see current dose amount, time of dose and dose information relative to previously stored, historical medicament administration events. The sensor mechanism can also provide sensing capabilities in or with respect to infusion devices to allow for early detection of device or component failures, medication errors and compliance. This allows for real-time detection and feedback to the user for infusion site failures and ensures improved safety, both of which have previously been unmet needs for users of conventional self-administered drug delivery devices.

Illustrative embodiments of the present invention can be implemented, at least in part, in digital electronic circuitry, analog electronic circuitry, or in computer hardware, firmware, software, or in combinations of them. The sensor mechanism that includes the electronics module or communicates with a separate electronics module and its respective components, includes a number of components, namely, a battery and signal processing unit can be implemented through application of one or more computer program products, i.e., a computer program tangibly embodied in an information carrier, e.g., in a machine-read able storage device or in a propagated signal, for execution by, or to control the operation of data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

Portions of the present disclosure can also be embodied as computer-readable codes on a computer-readable recording medium. The computer readable recording medium is any data storage device that can store data which can thereafter be read by a computer system. Examples of the computer-readable recording medium include, but are not limited to, read-only memory (ROM), random-access memory (RAM), CD-ROMs, magnetic tapes, floppy disks, and optical data storage devices. The computer readable recording medium can also be distributed over network-coupled computer systems so that the computer-readable code is stored and executed in a distributed fashion. Also, functional programs, codes, and code segments for accomplishing the present disclosure can be easily construed as within the scope of the invention by programmers skilled in the art to which the present disclosure pertains.

Method steps, processes or operations associated with the sensor mechanism and/or portable devices interfacing with the electronics module of the present disclosure operating in conjunction with a signal processing unit (processor) or controller or microcontroller associated with the sensor mechanism, or the medical device, or a user portable device (e.g., a handheld user device such as a smartphone, laptop, PDM and the like), can be performed by one or more programmable processors executing a computer program to perform functions of the invention by operating on input data and generating an output. Method steps can also be performed by, and an apparatus according to illustrative embodiments of the present invention, can be implemented as, special purpose logic circuitry, e.g., an FPGA (field programmable gate array) or an ASIC (application-specific integrated circuit).

Processors suitable for the execution of one or more computer programs associated with the present disclosure include, by way of example, both general and special purpose microprocessors, and any one or more processors of any kind of digital computer. Generally, a processor will receive instructions and data from a read-only memory or a random-access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example, semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be supplemented by or incorporated in special purpose logic circuitry.

The following list of medical devices, although not exhaustive, can benefit by the inclusion of the sensor mechanisms of the present disclosure:
injection devices, including reusable and disposable designs, needle or needleless,
manually driven by a user or triggered to automatically perform medicament delivery;
pumps that deliver medicament continuously or through intermittent bolus amounts;
inhalers, both dry powder and pressurized liquid atomizers;
osmotic delivery devices;
wearable patches; and
implanted biosensors and drug delivery devices.

As described in more detailed below, one possible medical device to benefit from the use of the sensor mechanism described herein is a pen-shaped injection device, such as those used to dispense insulin or fertility drugs, which historically have been strictly mechanical devices. There is growing interest in incorporating electronic functionality in these self-injection pens to monitor, track and accurately measure the above-mentioned parameters relating to dose setting and dose delivery. It is also of interest to incorporate wireless transmission of collected information relating to those parameters to the cloud to assist physicians in monitoring injection parameters. Solutions should be low cost, especially for the disposable pen market, must be accurate and should not require substantial modification of the existing pen mechanics.

One possible pen-type injection device that can be configured to incorporate a sensor mechanism is one that is capable of variable, user settable, multiple doses from a single container of medicament, where the container is preferably a cartridge. Examples of such devices are described in U.S. Pat. No. 8,512,296, U.S. Pub. No. 2018/0001031 and U.S. Ser. No. 15/649,287, filed Jul. 13, 2017, the contents of each of these patent applications are fully incorporated by reference in this application. Variants of the type of injection device described in U.S. Pat. No. 8,512,296 are also disclosed in international publications WO 2017/054917 A1 and WO 2013/117332 A1, the contents of each of these publications are fully incorporated by reference into this application.

The injection device can be reusable meaning that the container of medicament is replaceable through partial disassembly and resetting of the injection device, for example by replacing an empty cartridge with a full cartridge and retracting a piston rod back into a dose setting mechanism. In a reusable device, a cartridge holder is removed from the proximal end of the dose setting mechanism and the old empty cartridge is replaced by a new full cartridge and the cartridge holder is reattached to the dose setting mechanism. In a disposable injection device, the cartridge holder is permanently attached to the dose setting mechanism and once the cartridge of medicament is empty, the entire injection device is disposed of.

This disclosure describes in detail just one possible application of the use of the sensor mechanism that is integrated into an injection pen to measure, record and report parameters relating to dose setting and dose delivery. As stated, in one embodiment an electronics module can be removably attached to the pen that communicates with the first, second and/or third sensors that is then processed into a set dose and a delivered dose of medicament. This measured motion of the one or more ring shaped sensors is directly proportional to the relative movement of two component parts in the injection device, which in turn can be directly correlated to a dose of medicament set by the user and/or a dose of medicament delivered during the injection procedure.

The determination of dosage utilizing the sensor mechanism, as described in the present disclosure, is applicable to a wide variety of injection device designs, provided that at least one component of the dose setting and delivery mechanism moves during dose delivery. This change in position of the one or more sensors that is caused by linear or rotational movement can be directly proportional to an amount of medicament that would be expelled from the container of medicament if the injection procedure was fully carried out. Possible dose delivery components that move linearly are described in more detail below. These components can be a dose dial sleeve or a piston rod that translates proximally relative to the outer housing during dose delivery. Preferably, a linear sensor is fixedly attached to one or both of these components either through adhesion to an outside surface or incorporated in the components through co-molding.

As mentioned, an electronic module can constitute a separate or integral measuring device that collects, computes and records data derived from interrogating one or more of the sensors. If a separate electronics module is used, then it is desirable to configure the module as attachable, removable and reusable relative to the medical device of the present disclosure. By having a separate and reusable electronics module allows the medical device, for example an injection device, to be manufactured economically in a “ready state”, meaning ready for attachment of the electronics module. Further details of the separate electronics module are disclosed below. Alternatively, the electronics module can be integral, i.e., not a separate, removable part of the sensor mechanism contained within the injection device.

The sensors and electrical leads or conductors can be glued, press-fit, clamped, screwed, or otherwise physically attached to one or more selected components of the medical device. Alternatively, some sensors, for example the linear sensor, could be made integral to selected components of the medical device by co-molding one or more parts of such a sensor when the selected component is manufactured in the first instance. Such a manufacturing process is sometimes referred to a “two-shot” molding process. Co-molding allows for economically efficient manufacturing, especially when the medical device, such as an injection device or an inhaler, is intended as a disposable device, meaning that the container of medicament is sealed within the device and once all of the medicament has been expelled, usually through repeated injections or inhalations of the same or different doses, the medical device is then discarded. In other words, in such a disposable device there is no mechanism or means to remove an empty container of medicament or to reset the device to insert a new filled container of medicament.

### BRIEF DESCRIPTION OF DRAWINGS

The invention will be explainer in more detail hereinafter with reference to the drawings.

FIG. 1 illustrates a perspective illustration of one possible complete medicament delivery device that can incorporate a sensor mechanism of the present disclosure, where the cap of device is removed allowing attachment of a pen needle to the cartridge holder and showing an exploded view of the device;

FIG. 2 illustrates one possible logic flow diagram directed to an injection device having the sensor mechanism of the present disclosure;

FIG. 3 illustrates another possible logic flow diagram directed to an injection device having another embodiment of sensor mechanism of the present disclosure;

FIG. 4 illustrates a schematic representation of needed system architecture to allow the logic flow diagram of FIG. 3,

FIGS. 5A and 5B illustrate two perspective views of one possible embodiment of a rotating ring sensor for use in the sensor mechanism of the present invention;

FIG. 6 illustrates a perspective view of a possible embodiment of a stationary ring sensor for use in the sensor mechanism of the present invention;

FIG. 7 illustrates a cross-sectional view of an injection device containing an embodiment of the sensor mechanism of the present disclosure;

FIG. 8 depicts a schematic representation of the electrical signals generated by the sensor mechanism during dose setting and dose cancellation or correction;

FIGS. 9A and 9B illustrate two perspective views of one possible embodiment of one rotating ring sensor attached to a ring aligner and two stationary ring sensors for use in the sensor mechanism of the present invention;

FIG. 10 illustrates a cross-sectional view of an injection device containing the embodiment of FIGS. 9A and 9B of the sensor mechanism of the present disclosure;

FIG. 11 illustrates a perspective blown up view of the device and sensor mechanism of FIG. 10;

FIG. 12 is a schematic representation of yet another embodiment of the sensor mechanism of the present disclosure;

FIGS. 13A and 13B illustrate two perspective views of injection device incorporating the embodiment of the sensor mechanism of the present invention as depicted in FIG. 12;

FIG. 14 illustrates one possible embodiment of a reusable electronics module;

FIG. 15 and FIG. 16 illustrate the device of FIG. 1 having the electronics module of FIG. 13 releasably attached to the injection device of FIG. 1 where the display shows a zero-dose setting and where the display shows that a dose of 30 IU has been set, respectively; and

FIGS. 17A and 17B illustrate schematic representations of yet another embodiment of the sensor mechanism of the present disclosure having a dedicated EOD Switch in the open and closed position, respectively;

FIGS. 18A and 18B illustrate transparent perspective sections of one possible embodiment of the EOD Switch of the sensor mechanism of the present disclosure, implemented as conductive parts of or in the device, in the closed and open position, respectively;

FIG. 19 illustrates a transparent perspective section of another possible embodiment of the EOD Switch of the sensor mechanism of the present disclosure;

FIG. 20 illustrates an exploded view of parts of the sensor mechanism shown in FIG. 19;

FIG. 21 illustrates an insulating carrier of the sensor mechanism of FIG. 20;

FIG. 22 illustrates a second sensor element of the sensor mechanism shown in FIG. 19 positioned within an inset of a sleeve of the injection device shown in FIG. 19;

FIG. 23 illustrates the carrier of the sensor mechanism shown in FIG. 19 positioned on a support member;

FIG. 24 illustrates the sensor mechanism shown in FIG. 19 together with a knob of an injection device;

FIG. 25 illustrates the sensor mechanism and the knob shown in FIG. 24 from another perspective;

FIG. 26 illustrates a generation of quadrature sensor signals with the sensor mechanism shown in FIG. 19;

FIG. 27 illustrates a further embodiment of an injection device that has a rotary dose setting sensor and a linear dose delivery sensor, whereby a knob of the injection device is positioned in a dose setting position;

FIG. 28 illustrates the further embodiment of the injection device shown in FIG. 27 with the knob being positioned in a dose delivery position; and

FIG. 29 illustrates a schematic representation of an exploded view of a dosing mechanism of the injection device shown in FIGS. 7 to 28.

### DETAILED DESCRIPTION

In the present application, the term “distal part/end” refers to the part/end of the device, or the parts/ends of the components or members thereof, which in accordance with the use of the device, is located the furthest away from a delivery/injection site of a patient. Correspondingly, the term “proximal part/end” refers to the part/end of the device, or the parts/ends of the members thereof, which in accordance with the use of the device is located closest to the deliveryAnjection site of the patient.

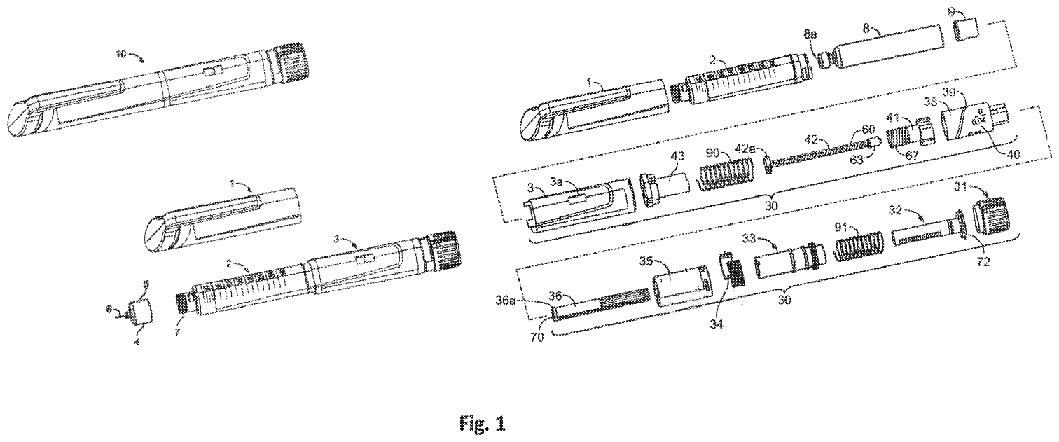

One example of a medical device that can incorporate one of a number of possible sensor mechanisms of the present disclosure is a pen-type injector device 10, as shown in FIG. 1. The complete injection device 10 is illustrated as well as an exploded view of the device, which is presented in the zero-dose state as indicated by indicia 40 showing a zero through the window 3a of housing 3. The device 10 with cap 1 removed exposes the cartridge holder 2 and the proximal needle connector 7 that is configured for a pen needle 4 that is typically attached to the needle connector 7 through a snap fit, thread, Luer-Lok, or other secure attachment with hub 5 such that a double ended needle cannula 6 can achieve a fluid communication with medicament contained in cartridge 8 positioned within cartridge holder 2. The cartridge 8 is sealed at the proximal end by septum 8a and with a sliding piston 9 (or piston disc, bung, stopper) at the opposite distal end.

The pen-type injection device of FIG. 1 has a sleeve 35 that translates in a longitudinal direction during dose setting, dose correction and dose delivery. A dose is set with dose setting mechanism 30 through rotation of dose knob 31, which causes sleeve 35 to move linearly in the distal direction. A dose is delivered by pushing on the end of the dose knob 31 in the opposite or proximal direction. This in turn causes sleeve 35 to move linearly back into the dose setting mechanism in the proximal direction.

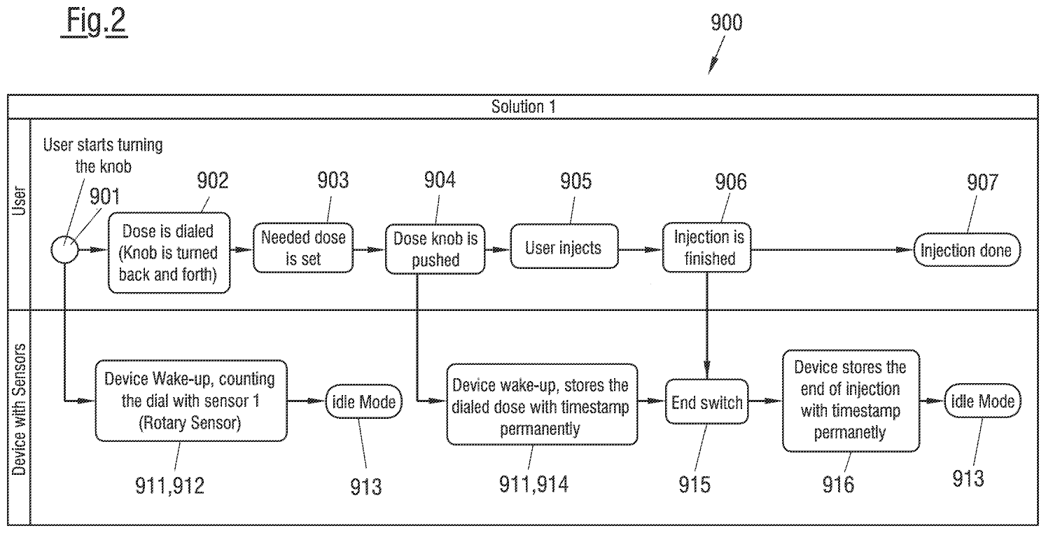

FIG. 2 schematically shows operational flow diagrams for two possible embodiments of the present disclosure. FIG. 2 illustrates a logic diagram for an embodiment that only involves the use of one or more rotary dose setting sensors, such as ring sensors, to detect a dialed dose and/or cancellation of a dialed dose to arrive at a set dose immediately prior to dose delivery in the medical device of FIG. 1.

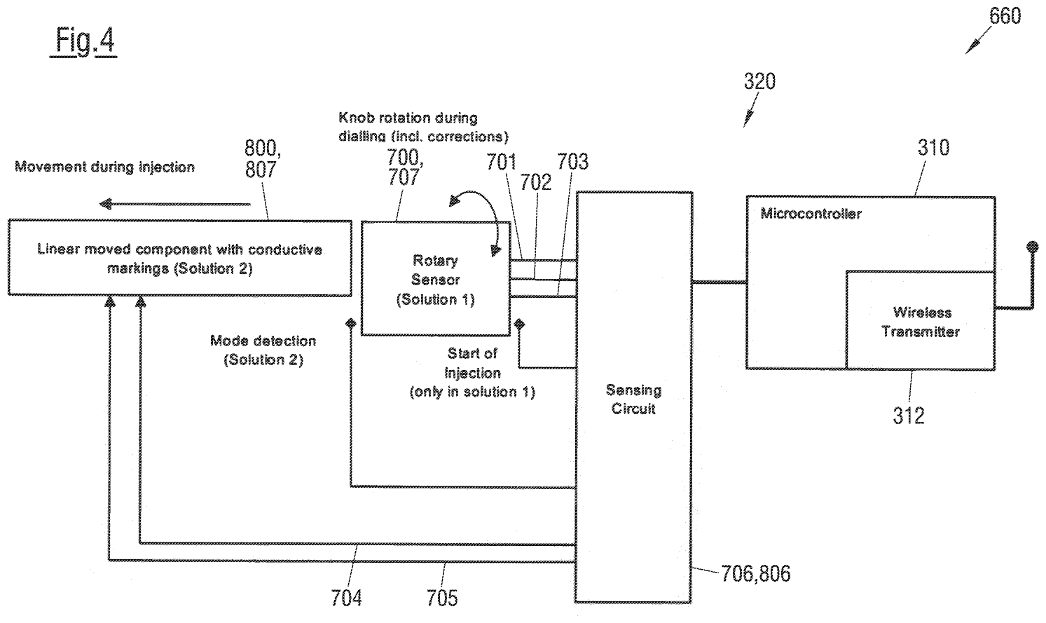

FIG. 4 illustrates a logic flow diagram for the use of another sensor mechanism that employs both a rotary dose setting senor, such as ring sensors, and a linear sensor, where the linear sensor can determine the start of the delivery of the set dose of medicament, the actual amount of medicament delivered, the time to complete the delivery, the end of delivery and whether the delivery was interrupted. FIG. 4 shows the needed electromechanical and electronics components to fulfil the illustrated flow.

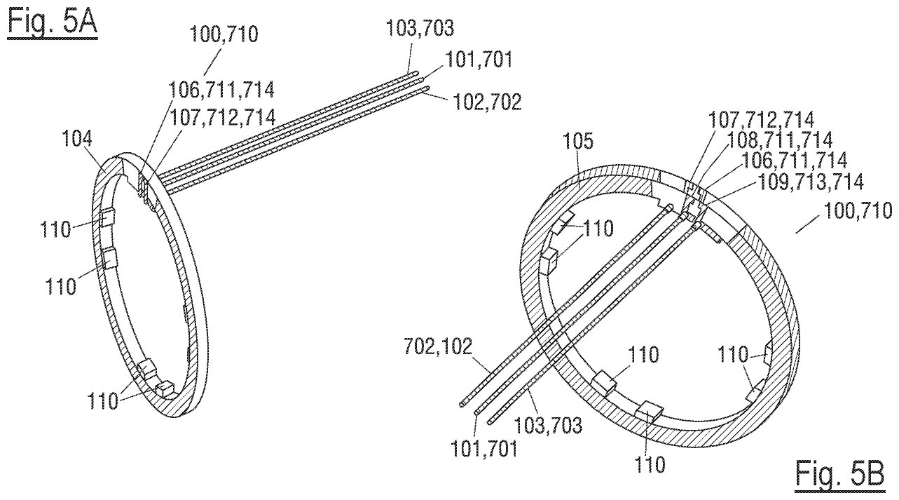

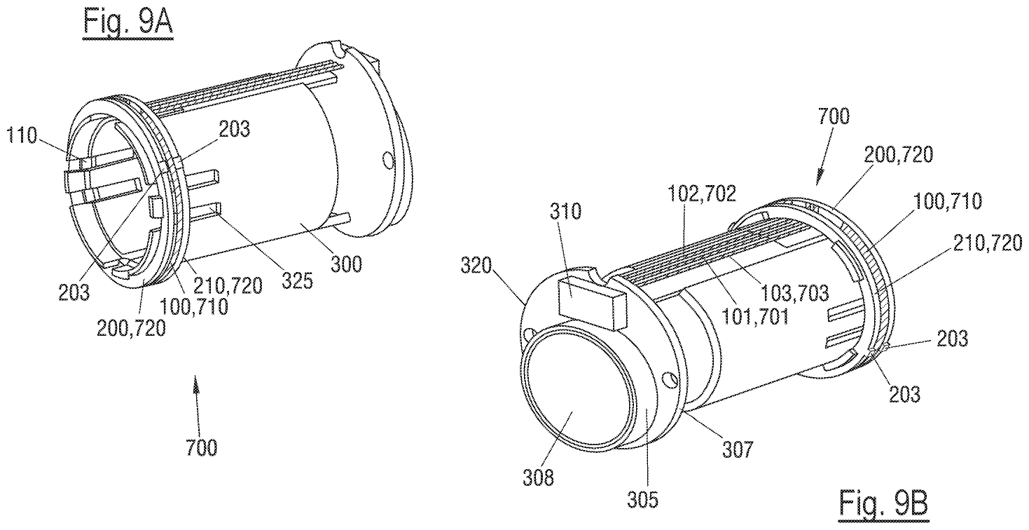

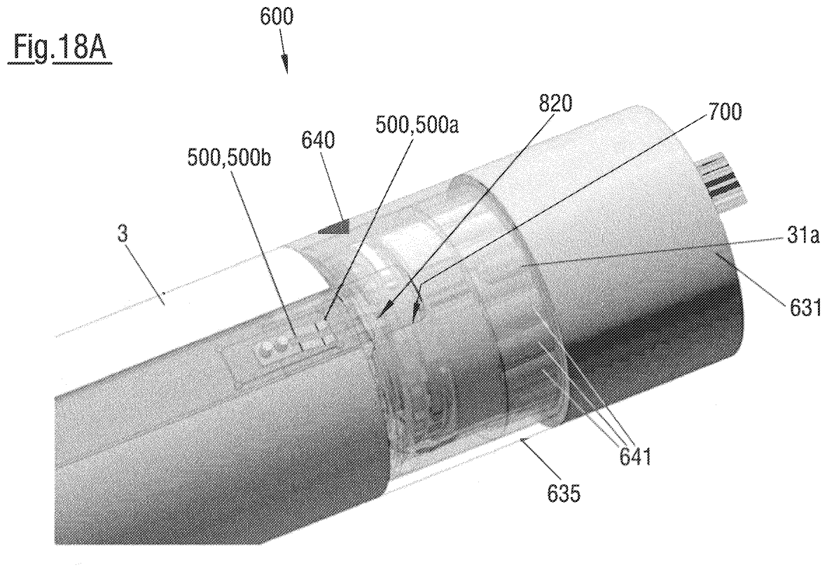

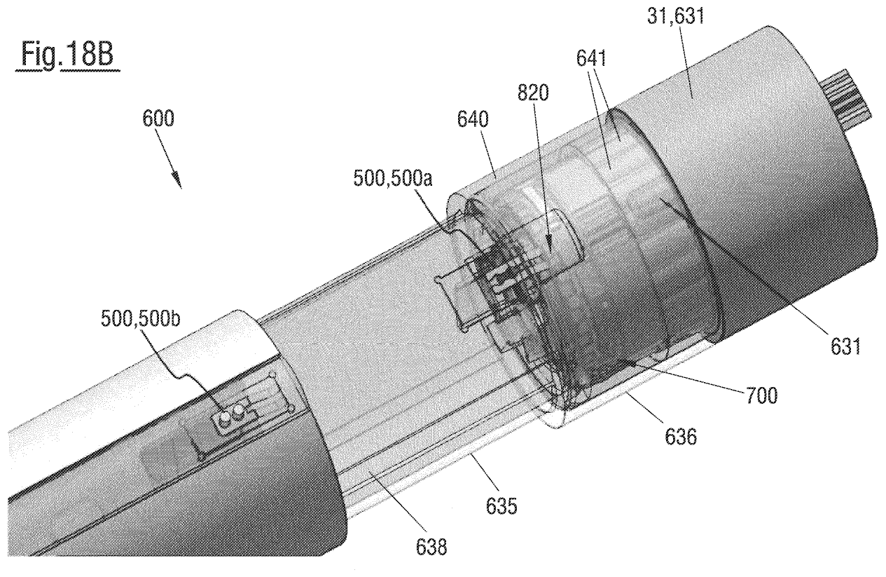

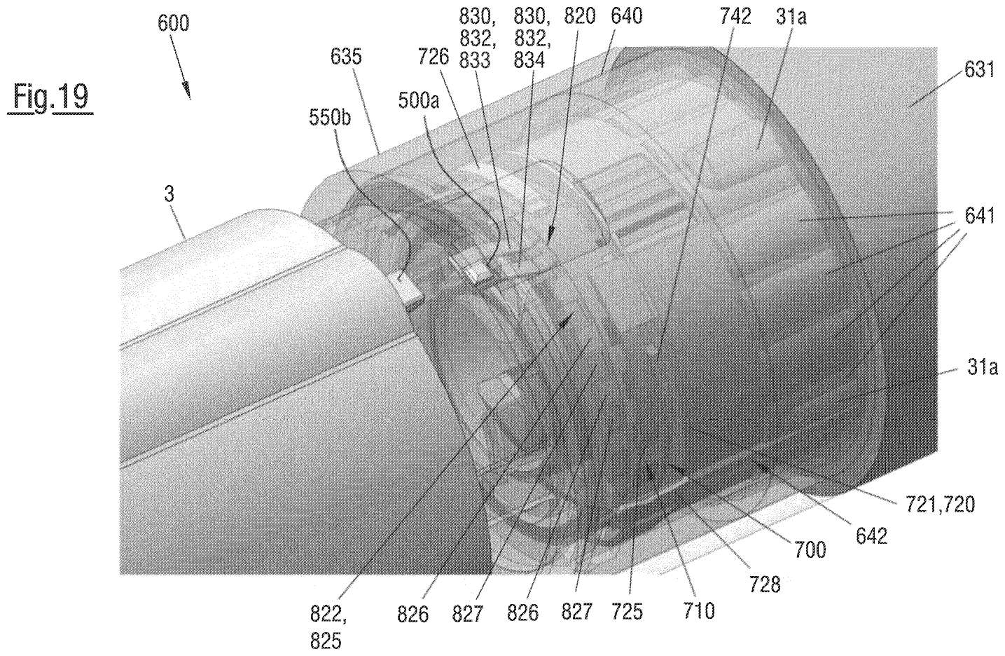

FIGS. 5A and 5B illustrate one embodiment of a ring sensor 100 of the present disclosure that is configured for attachment to a ring aligner 300 (see FIGS. 9A and 9B). Ring sensor 100 is designed to include electrical leads 101, 102 and/or 103 that are electrically connected to a respective contact point 106, 107, 108, and/or 109, wherein a first contact point 106 is electrically connected to a first electrical lead 101, a second contact point 107 is electrically connected to a second electrical lead 102, a third contact point 108 is electrically connected to the first electrical lead 101 and a fourth contact point 109 is electrically connected to a third electrical lead 103. These contact points are provided on the end faces 104 and 105 of the ring sensor 100. In some cases, where only a dose dealing and not dose correction is to be detected, only two contact points and two electrical leads are required. For example, only electrical leads 101 and 102 are needed and the corresponding contact points 106 and 107. Such would be the case where the sensor mechanism includes only two ring sensors, one rotating ring sensor such as sensor 100 and one fixed ring sensor such as sensor 200. Although the embodiment as shown FIGS. 5-11 illustrates the sensors 100, 200 and 210 as rings, these sensors could be configured as barrel-like structures similar to what is illustrated in FIGS. 18A, 18B, and 19 as part number 700. The use of such barrel or tube or cylinder type sensors will typically lead to a use of the contacts in radial direction.

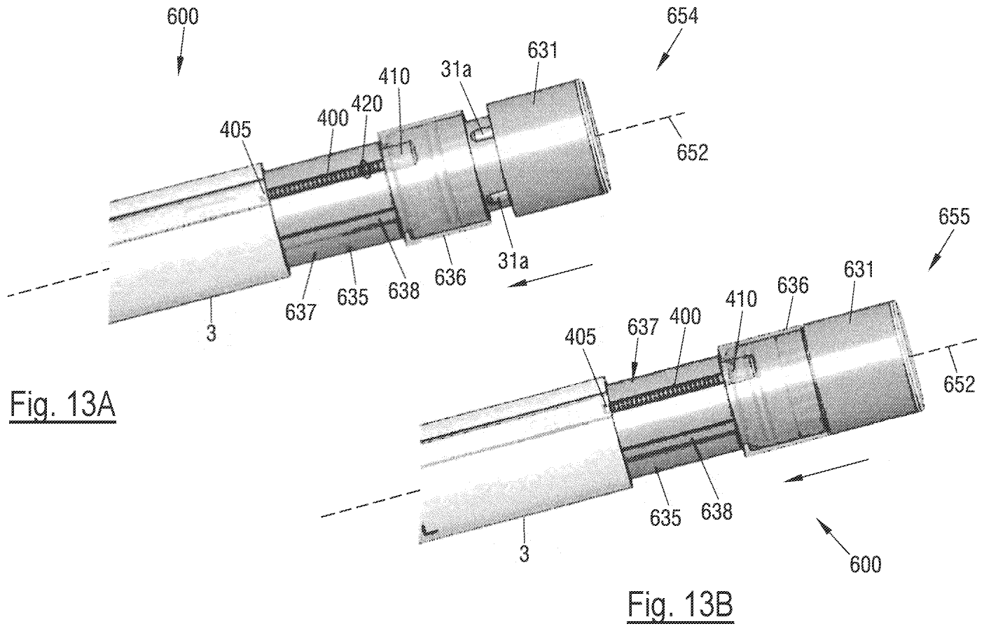

The injection device 10 shown in FIG. 1 is also described in detail in documents U.S. Pub. No. 2018/0001031 and U.S. Ser. No. 15/649,287. While the injection device 10 can also feature the sensor mechanism comprising the ring sensors 100, 200, 210, the integration of the sensor mechanism into an injection device will be described in the following in connection with a further injection device 600, which is shown, inter alia, in FIGS. 13A and 13B. The further injection device 600 incorporates a dosing mechanism for dose setting and dose expelling that is described in U.S. Pat. No. 8,512,296, WO 2017/054917 A1 and WO 2013/117332, wherein the description of the dosing mechanism in these documents is incorporated by reference into the present disclosure, in particular as far as configuration and relative maneuverability of a housing, an injection sleeve, a metering element, a piston rod and a rotary knob of these devices are concerned.

As can be seen from FIGS. 13A and 13B, the further injection device 600 comprises, like the injection device 10, a sleeve 635 that is axially movable and rotationally fixed with respect to a housing 3 during both dose setting and dose delivery. With the further injection device 600, the sleeve 635 forms an injection sleeve as described, inter alia, in U.S. Pat. No. 8,512,296. In accordance with the dose setting knob 31 of the injection device 10, the further injection device 600 comprises a rotatable control element for dose setting that is located at a distal end of the sleeve 635. This control element forms a rotary knob 631 as described, inter alia, in U.S. Pat. No. 8,512,296B1.

FIGS. 13A and 13B depict the further injection device 600 in a state, in which a dose has been set by the user by rotating the knob 631 in the clockwise direction. During dose setting, the sleeve 635 and the knob 631 move in the distal direction with respect to the housing 3 along a longitudinal axis 652 of the further injection device 600. As long as no dose is set (zero dose setting), the sleeve 635 is located at its most proximal position with respect to the housing 3. In this position, a radially protruding distal projection 636 of the sleeve 635 abuts at the distal end of the housing 3 and a proximal shell portion 637 of the sleeve 635 is completely located inside the housing 3. During dose setting, the shell portion 637 moves axially out of the housing 3. Rotation of the sleeve 635 with respect to the housing 3 is prevented by a positive locking mechanism. The positive locking mechanism comprises the longitudinal grooves 638 located at the outer surface of the shell portion 637 and corresponding longitudinal splines located on the inner surface of the housing 3. With other embodiments, such as the embodiment described in U.S. Pat. No. 8,512,296 B1, the grooves 638 can also be located on the housing 3 and the protrusions on the shell portion 637.

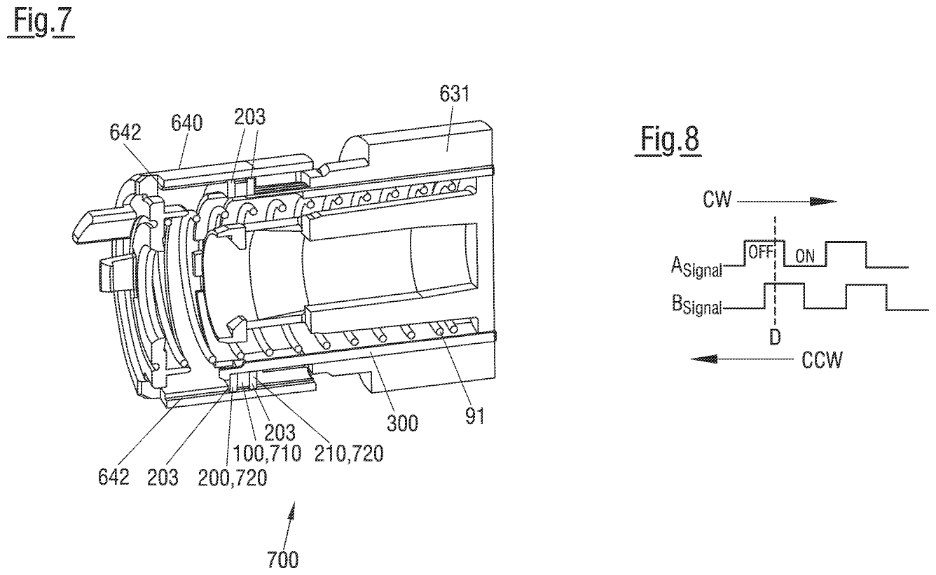

The knob 631 is axially movable with respect to the sleeve 635 in the proximal direction from a dose setting position 654 into a dose delivery position 655. In the dose setting position 654, into which the knob 631 is biased by a biasing member 91 (shown in FIG. 7), the knob 631 is located in its most distal position and is rotatable with respect to the sleeve 635 and the housing 3. In the dose delivery position 655, the control element 631 abuts against the distal end of the sleeve 635. In this position, the knob 631 is rotationally fixed with respect to the sleeve 635 and the housing 3 via the coupling 31a.

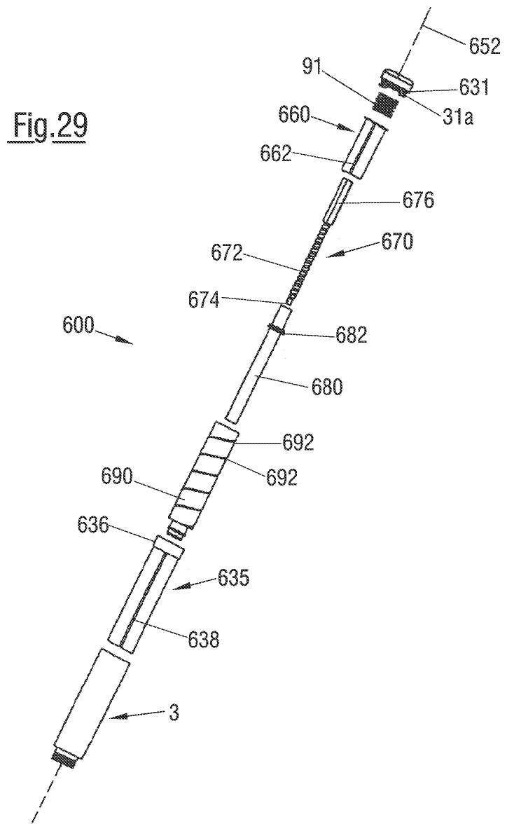

FIG. 29 schematically depicts an exploded view of the dosing mechanism of the further injection device 600, which is configured to be actuated for dose setting and dose delivery. The dosing mechanism comprises the housing 3, the sleeve 635, the knob 631, the biasing member 91, which is configured as a compression spring, a driver 660, a piston rod 670, which is surrounded by a tube 680, and a metering element 690.

The sleeve 635 is configured as a generally cylindrical hollow member that is received within the housing 3 and that is axially movable and rotationally fixed with respect to the housing 3 via the positive locking mechanism comprising the longitudinal grooves 638. The metering element 690 is mounted rotationally movable and axially fixed with respect to the housing 3. It is surrounded by the sleeve 635 and engages with the sleeve 635 via an external thread 692, which couples the rotational movement of the metering element 692 to the axial movement of the sleeve 635. The piston rod 670 is coupled to the metering element 690 via an external thread 672 so that rotation of the metering element during rotational fixation of the piston rod 670 moves the piston rod 670 in the axial direction. The proximal end 674 of the piston rod 670 is configured to abut against a piston that urges medicament out of a cartridge connected with the housing 3.

The distal part 676 of the piston rod 670 is slideably received within the tube 680, whereby the distal part 676 is axially movable and rotationally fixed with respect to the tube 680. The tube 680 is both rotationally and axially fixed to the knob 631 and is surrounded by the driver 660. The driver 660 is slideably received within the metering element 690, so that it is axially movable and rotationally fixed with respect to the metering element 690. Thereby, the driver 660 engages with the inner lateral surface of the metering element 690 via grooves 662, which receive corresponding protrusions at the inner surface of the metering element 690. With other embodiments of the further injection device 600, such as the embodiment described in U.S. Pat. No. 8,512,296 B1, the grooves 662 can also be configured at the inner surface of the metering element 690 and the driver 660 can comprise the corresponding protrusions. Furthermore, the driver 660 is, at its distal end, rotationally movable and axially fixedly connected to the sleeve 635.

The tube 680 is longitudinally movable with respect to the driver 660 between a proximal end position and a distal end position, whereby movement of the tube 680 beyond the end positions is inhibited by hard stops configured between the tube 680 and the driver 660. The tube 680 has at its outer surface an external spline set 682 that engages with a corresponding spline set at the inner surface of the driver 660 if the tube 680 is located at its distal end position and that is disengaged from the corresponding spline set if the tube 680 is located at its proximal end position. Therefore, the tube 680 is rotationally fixed with respect to the driver 660 when being located at its distal end position and rotationally movable with respect to the driver 660 when being located at its proximal end position. The spline sets thereby form a releasable coupling between the tube 680 and the driver 660.

During dose setting, the knob 631 and the tube 660, which is fixedly connected to the knob 631, are biased by the biasing member 91 in the distal direction. Thereby the biasing member 91 acts between the knob 631 and the driver 660. This closes the releasable coupling between the tube 680 and the driver 660, so that both the metering element 690, which is rotationally fixed to the knob 631 via the driver 660 and the closed coupling to the tube 680, and the piston rod 670, which is rotationally fixed to the knob 631 via the tube 680, rotate in unison when the knob 631 is rotated during dose setting. Therefore, the piston rod 670, despite being threadedly connected to the metering element 690, does not move relative to the metering element 690 and the housing 3. However, rotation of the metering element 690 during dose setting urges the sleeve 635 and the knob 631 out of the housing 3 via the threaded connection between the metering element 690 and the sleeve 635.

When the knob 631 is pushed against the sleeve 635 to initiate dose delivery, the tube 680 moves from its distal end position to its proximal end position and the releasable coupling between the tube 680 and the driver 660 is released. Therefore, the metering element 680 and the driver 660 become rotationally movable with respect to the knob 631. At the same time, the knob 631 becomes rotationally fixed with respect to the sleeve 635 and the housing 3 via the coupling 31a. This also rotationally fixes the piston rod 670 with respect to the housing 3 via the tube 680, the knob 631 and the coupling 31 to the sleeve 635.

If the knob 631 and the sleeve 635 are now further pushed in the proximal direction with respect to the housing 3, the axially moving sleeve 635 induces rotary motion of the metering element 690 via the thread 692. The rotating metering element 690 then drives the now rotationally fixed piston rod 670 in the proximal direction with respect to the housing 3 to expel medicament from the attached container.

When integrating the sensor mechanism into the injection devices 10, 600, the fixed ring sensor 200 is rotationally fixed relative to the housing 3 of the devices 10, 600. As mentioned, ring sensor 100 is connected to the ring aligner 300, which is rotationally fixed connected to the dose setting knob 31, 631 (see FIGS. 7 & 10). This connection of the sensor 100 with the aligner 300 is through the cooperation and/or engagement of one or more lugs 110 located on an inside surface of the ring sensor 100 with slots or cut-outs 325 on the outside surface of the ring aligner 300. This operative association of the ring sensor 100 with the ring aligner 300 and with the dose knob 31, 631 results in rotational fixation such that rotation of the dose knob 31, 631 during dose selection, or in the opposite direction during dose correction, simultaneously causes the ring aligner 300 and the rotatably fixed ring sensor 100 to also rotate. Because the ring sensor 200 and/or the ring sensor 210 are rotationally fixed relative to the housing 3, rotation of the dose knob 31, 631 causes relative rotation between the sensor 100 and the sensor 200. And, if the second stationary ring sensor 210 is part of the sensor mechanism, then there will be relative rotation between the sensor 100 and sensors 200, 210.

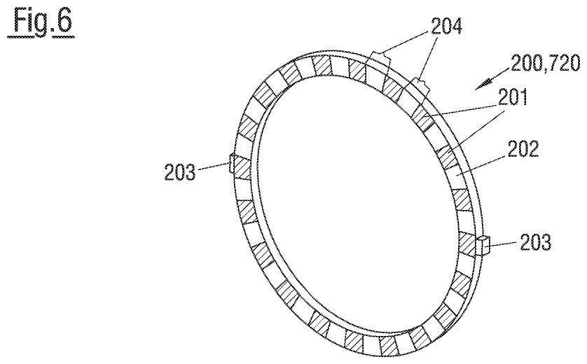

Relative rotation of the ring sensor 100 relative to either or both of the ring sensors 200, 210 will now be explained. As shown in FIG. 6, the sensors 200, 210 each have an end face 202 that contains a plurality of contact surfaces 201 spaced around the circumference of the end face 202. These contact surfaces 201 are comprised of electrically conductive materials, such as a metal, and are preferably equally spaced around the end face 202. The spaces 204 between each contact surface 201 are non-electrically conductive such that adjacent surfaces 201 are not in electrical communication with each other. Preferably, the spaces 204 are comprised of a non-conductive material. The sensor mechanism is constructed so that the ring sensor 100 is flush against the ring sensors 200 and/or 210. This flush arrangement is such that the contact points 106, 107, 108 and/or 109 will make electrical contact with the contact surfaces 201 as the sensor 100 rotates relative to the sensors 200, 210.

In an embodiment of the sensor mechanism where dose correction is to be measured and monitored, then the two ring sensors 200, 210 are positioned in a flush arrangement/engagement on either side of ring sensor 100 as illustrated in FIGS. 7, 9 & 10. In order to determine the direction of rotation of sensor 100, i.e., whether a dose is being set or a set dose is being cancelled, it is necessary to position the contact points 106, 107 in an offset relationship from contact points 108, 109. This offset relationship is clearly illustrated in FIGS. 5A and 5B and is schematically illustrated in FIG. 8 where CW (clockwise rotation) is the direction the sensor 100 is turned via dose knob 31, 631 to set a dose of medicament and CCW (counterclockwise) is the opposite direction indicative of cancelling or correcting a set dose, which can occur if a user inadvertently rotates the dose knob 31, 631 too far and over shoots a desired dose setting. Rotating in the CCW direction allows the user to reduce the inadvertently set high dose to a lower correct/desired dose. As indicated in FIG. 8, as the sensor 100 rotates relative to the two stationary sensors 200, 210, the offset contact points will make offset electrical contact with the contact surfaces 201. This is illustrated by signals A and B shown in FIG. 8, which will be received by the electronics module 320, which contains a battery 308 and a microcontroller 310. In the example illustrated, the battery 308 is located in a battery compartment 305 and accessible by a removable cap 306. Both the microcontroller 310 and battery 308 are positioned on, or are an integral part of, the ring aligner 300 such they rotate with sensor 100. The microcontroller 310 and the battery 308 are positioned on a support board 307, such as a printed circuit board, that is rotationally fixedly mounted at the distal end of the ring aligner 300.

In addition to the possible inclusion of three ring sensors (100, 200, & 210) in one embodiment of the sensor mechanism of the present invention, another embodiment could further include one or more linear sensors. In the case of the injection devices illustrated in FIGS. 1 and 13, the linear movement of the dose selector or sleeve 35, 635 is a result the outer surface of the sleeve 35, 635 having one or more of the longitudinal grooves 638 that are always engaged with longitudinal splines located on the inner surface of housing 3. This engagement prevents relative rotation between the dose selector or sleeve 35, 635 and the housing 3 but allows the dose selector or sleeve 35, 635 to move axially relative to the housing 3. With the injection device 10 shown in FIG. 1, the outer surface of the dose selector or sleeve 35 also has connecting cut-outs that permanently engage and lock with snap fits on the dose knob 31 such that the dose knob 31 is axially fixed to the dose selector or sleeve 35. These permanent snap fits allow the dose knob 31 to rotate relative to the dose selector or sleeve 35 during both dose setting and dose cancellation. The linear movement of the dose selector or sleeve 35 presents a viable component of the injection device 10 to include a fourth sensor, e.g., a linear sensor. It is also possible to include a linear sensor on the piston rod 42 shown in FIG. 1. With the further injection device 600 shown, inter alia, in FIGS. 13A and 13B, the dose knob 631 is axially movable with respect to the sleeve 635 from the dose setting position 654 into the dose delivery position 655. However, also with the further injection device 600, the sleeve 635 is rotationally fixed with respect to the housing 3 and moves linearly along the longitudinal axis 652 during dose delivery. Like the sleeve 35 of the injection device 10, also the sleeve 635 of the further injection device 600 can carry a linear sensor for sensing the delivery of the set dose.

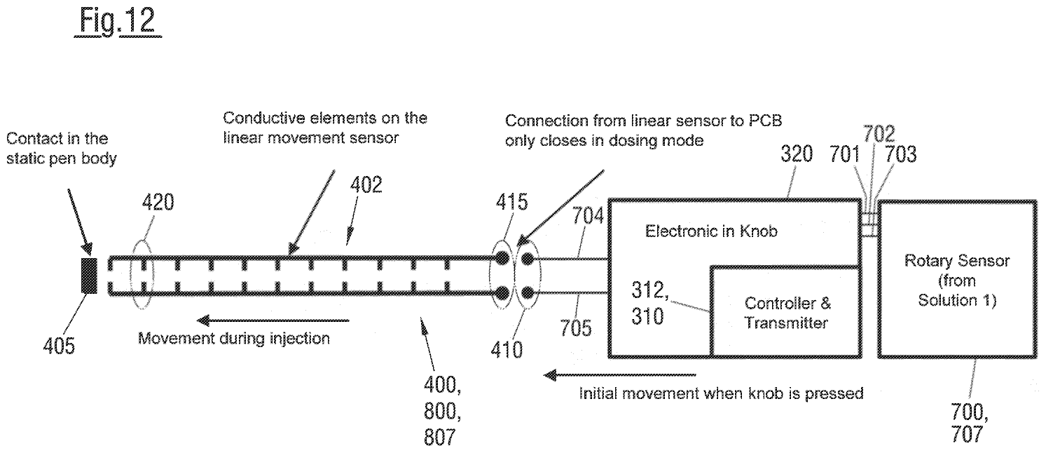

The use of a linear sensor 400 as part of the sensor mechanism is illustrated schematically in FIG. 12 and as part of the further injection device 600 in FIGS. 13A and 13B. If the injection device 10 carries the linear sensor 400, it can have a corresponding configuration. In general, as best illustrated in FIG. 12, the linear sensor 400 is only activated after a dose has been set. In other words, the linear sensor 400, which is positioned on sleeve 635, is not in electrical contact or communication with the electronics module 320 associated with the sensor rings 100, 200 and/or 210 during dose setting. As illustrated in FIGS. 12 & 13, when the knob 631 is in an extended position, which is the dose setting position 654, such that coupling 31a is open, i.e., not engaged with the housing 3 via the sleeve 635 such that the knob 631 can rotate relative to the housing 3, the contact pins 410 are not in electrical contact with the terminal ends 415 of the linear sensor 400. This represents an open circuit situation. When the dose setting knob 631 is in the extended position 654 the user is able to rotate the knob 631 to set or cancel a dose. Once the desired dose is set, the user will then push the dose setting knob 631 in the proximal direction to a forward position, which is the dose delivery position 655. This will also close the coupling 31a such that the knob 631 cannot be rotated relative to the housing 3. Once the knob 631 is pushed proximally to its forward position 655, the contact pins 410 will come into electrical contact with terminal ends 415 thus closing the circuit resulting in the linear sensor 400 becoming connected to the electronics module 320.

As the user continues to push the dose knob 631 proximally, this will begin the delivery (injection) of the set dose of medicament. Sleeve 635, which contains the linear sensor 400, will be driven axially in the proximal direction as the knob 631 is pushed by the user. This will cause the linear sensor 400 to move proximally relative to a static electrical contact 405 that is fixed to the housing 3, preferably located on the inside surface such that it can be in a sliding engagement with the linear sensor 400. One possible design and configuration of the linear sensor 400 is illustrated FIGS. 12 & 13, where pairs of opposing contacts 420 are equally spaced along the longitudinal axis of the linear sensor 400. As best illustrated in FIG. 12, when electrical contact is made between pins 410 and terminal ends 415 there is no continuity through the linear sensor 400 because the opposing pairs of contacts 420 are all open. However, as each pair of contacts 420 slides past the static contact 405 the circuit becomes closed and continuity is momentarily achieved until the circuit is opened again as the sleeve 635 and linear sensor 400 continue moving in the proximal direction. The electronics module 320 will monitor and record this opening and closing of the circuit as the sleeve 635 continues moving.

By making the spacing between the opposing contacts 420 directly proportional to a fixed amount of medicament, a determination of the amount of medicament actually delivered can be determined. Monitoring this movement of the linear sensor 400 can also be used to determine the time it took the user to inject the set dose of medicament and whether there was an interruption in the delivery or if there was delivery of less than the set dose of medicament. Likewise, the start of dose delivery can be determined as well as the end of dose delivery. In each case this information could be used to cause an audible signal to be emitted to inform the user of the status of dose delivery.

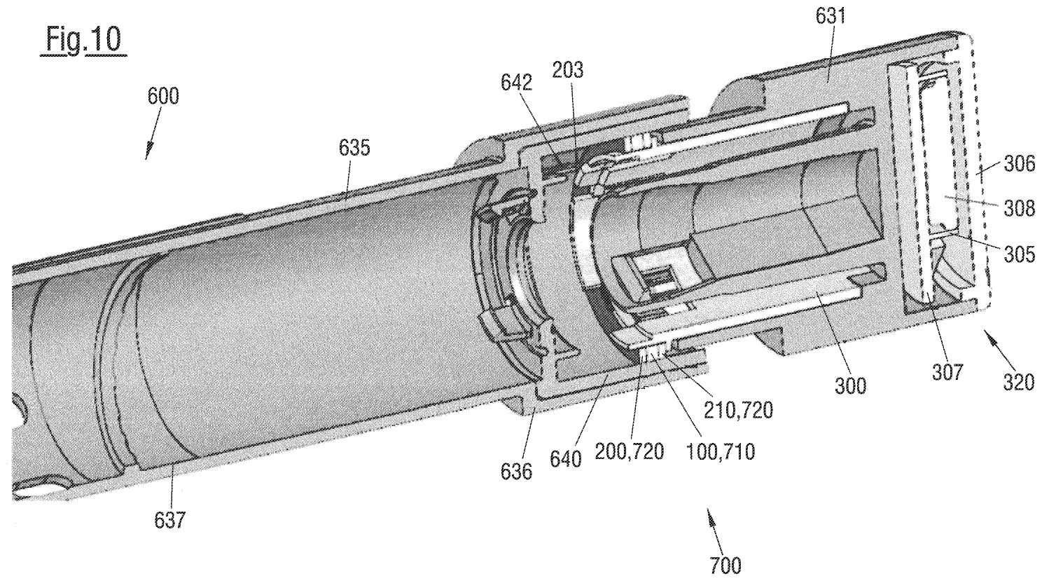

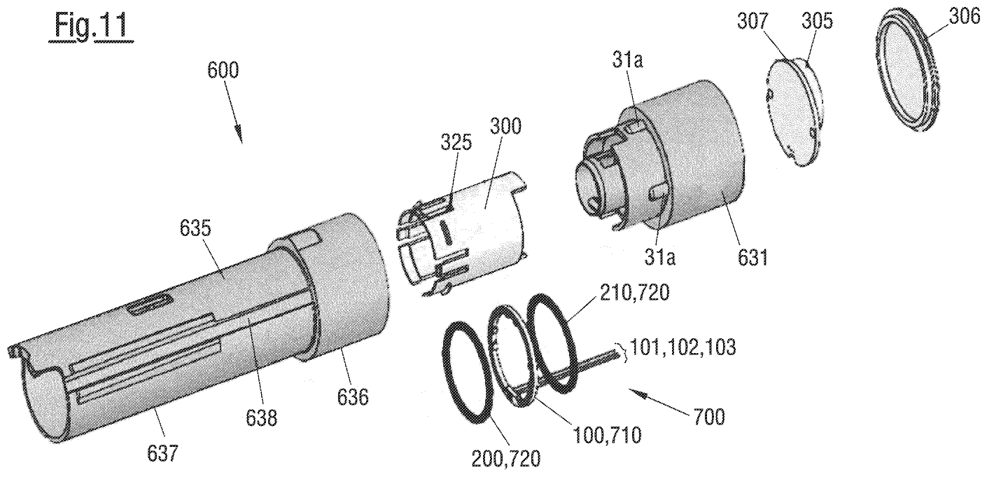

As stated, FIG. 1 shows just one possible design of an injection device 10 that allows for setting of one or more of the predetermined fixed doses through the interaction of a snap element 33 with the dose selector or sleeve 35, which could contain linear sensor 400 fixed to the outer surface. As mentioned, in both the injection device 10 and the further injection device 600, the linear sensor 400 could also be imbedded into the dose selector or sleeve 35, 635 during the manufacture of the dose selector or sleeve 35, 635 for example by co-molding. With the injection device 10 shown in FIG. 1, the dose knob 31 is pressed in the proximal direction during the initiation of the dose delivery procedure causing the dose knob 31 and the dose selector 35, along with the linear sensor 400, to move axially relative to the snap element 33. With the injection device 600 shown, inter alia, in FIGS. 10 and 11, the dose knob 631 is also pressed in the proximal direction during the initiation of the dose delivery procedure. However, this only causes the dose knob 31, along with the linear sensor 400, to move axially in the proximal direction, not the dose selector 35. With both injection devices, this initial movement disengages a splined connection and causes engagement of a different splined connection which prevents the dose knob 31, 631 from rotating relative to the housing 3 during dose delivery. The initial movement of the dose knob 31, 631 with respect to the dose selector 35, 635 could engage contact pins 410 with the terminal ends 415.

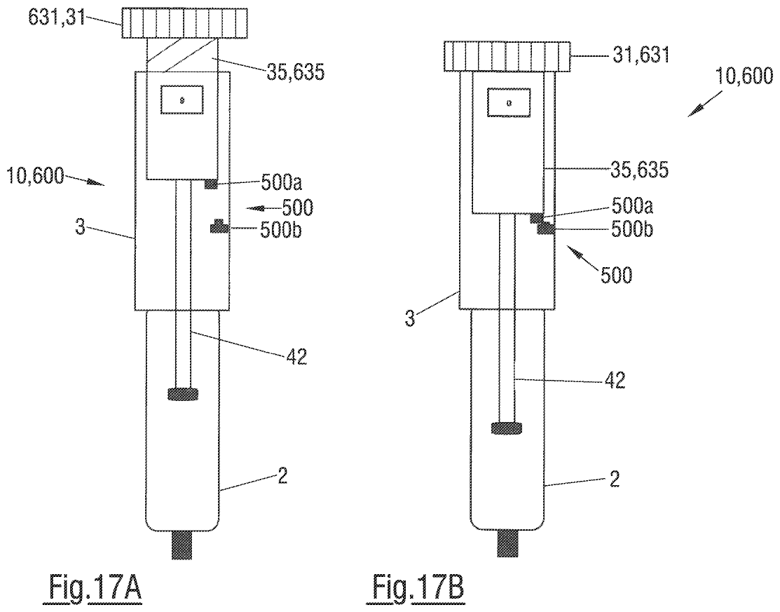

Turning next to FIGS. 17A to 19, the sensor mechanism of the present disclosure can also contain an end of dose delivery notification feature, preferably in the form of an EOD Switch 500. FIGS. 17 A and 17B schematically illustrate the medicament delivery devices 10, 600 having the dose setting knob 31, 631, a dose delivery mechanism, such as the dose delivery mechanism 30, the dose dial sleeve 35, 635 and the piston rod 42. The EOD Switch 500 can be formed from two separate components 500a and 500b, where EOD Switch part 500b is linearly fixed relative to housing 3. EOD Switch part 500a can be positioned on dose dial sleeve 35, 635 such that as the dose dial sleeve 35, 635 moves distally during dose setting the distance between parts 500a and 500b increases. As the set dose is being delivered the dose dial sleeve 35, 635 will move proximally until the two parts 500a and 500b connect with each other as illustrated in FIG. 17B thus closing the EOD Switch 500 and completing an electrical circuit that is monitored by the microcontroller 310. This connection of parts 500a and 500b can only be achieved when the set dose (shown in FIG. 17A as “9”) has been completely delivered by device 10, 600.

FIGS. 18A, 18B and 19 show different possible embodiments of EOD Switch 500, each of which is part of the sensor mechanism and are configured as a mechanical switch or physical interaction of electrical contacts. As mentioned, the status of the EOD switch 500 is monitored by the microcontroller 310 and that status data can be transmitted via a wireless interface 312 to an external device (e.g., smart phone, tablet, docking station, etc.). The EOD Switch status can also be transmitted to mobile network. Part 500a of the EOD Switch 500 can be electrically connectable to the microcontroller 310 and part 500b can be in a fixed position with respect to the housing 3 and located proximally from the linearly movable dose dial sleeve 35, 635, for example proximally from the distal projection 636 of the sleeve 635. In general, part 500b is positioned proximally from part 500a. As illustrated in FIGS. 17A and 18A, the EOD Switch 500 is placed in such that part 500a is located on the dose dial sleeve 35, 635 and will contact part 500b, which is positioned at the housing 3, at exactly the same time when the dose dial sleeve 35, 635 reaches and is at zero dose position. During the setting of a dose (see FIGS. 17B, 18B and 19), the dose dial sleeve 35, 635 moves linearly relative to the device housing 3 in the distal direction away from the part 500b of the EOD Switch 500 and simultaneously causes the switch 500 to move to an open position, i.e., opening the electrical circuit. In some drug delivery device designs the dose dial sleeve 35, 635 moves both rotationally and linearly during dose setting and dose delivery. With other embodiments, such as with the delivery devices 10, 600, the sleeve 35, 635 is mounted rotationally fixed with respect to the housing 3 and moves only axially during dose setting and dose delivery.

During the process of delivering the set dose of medicament, the dose dial sleeve 35, 635 is moved linearly relative to the device housing 3 until part 500a of the EOD Switch 500 connects with part 500b, thus closing the EOD Switch 500. The predetermined position of the two parts of the EOD Switch 500 are designed and configured such that the closing of the EOD Switch 500 coincides exactly with the complete delivery of the set dose of medicament and only happens when the dose dial sleeve 35, 635 is at zero position. If the drug is not completely delivered the EOD Switch 500 is not closed and that information is communicated to the external device and ultimately to the user of the device. Once the dose is completely injected the EOD Switch 500 will close and the status of the switch 500 is read by the microcontroller 310 and the dose can be calculated, along with the time of dose delivery, and this data can be transmitted via the wireless interface 312 to the external device or directly into a mobile network.

Part of the dose setting mechanism of most pen-type injectors, including devices 10, 600, is a piston rod 42 as illustrated in FIG. 1 for the injection device 10. In those device designs where the piston rod 42 does not rotate during dose delivery, such as the injection device 10 and the further injection device 600, there is the possibility that the linear sensor 400 or another linear sensor can be applied to or incorporated within the outer surface of the piston rod 42. With the injection device 10, the piston rod 42 is rotationally fixed with respect to the housing 3 during both dose setting and dose delivery. With the further injection device 600, the piston rod is rotationally movable with respect to the housing 3 during dose setting and rotationally fixed with respect to the housing 3 during dose delivery. Piston rods 42 that do not move during both dose setting and dose delivery usually have a non-circular cross-section and have two flat surfaces that are designed to prevent the piston rod 42 from rotating yet allow it to move linearly in the proximal direction. A preferred method to measure the translation of the piston rod 42 is to apply or otherwise add a linear sensor along the length of the existing piston rod design in a similar fashion as that described to monitor and measure the sleeve 35, 635.

Returning to the specifics of the dose setting mechanism 30 of device 10, a nut 36 and a clutch 32 are permanently splined to each other during assembly of the dose setting mechanism 30 through a splined connection. The splined connection ensures that clutch 32 and nut 36 are always rotationally fixed to each other during both dose setting and dose delivery. This splined connection also allows the clutch 32 and the nut 36 to move axially relative to each other. The sliding connection is necessary to compensate for the difference in the pitch of the thread between nut 36 and the outer surface of the piston rod 42 and the pitch of the thread between a dose sleeve 38 and body 3. The thread between a driver 41 and a piston guide 43 has basically the same pitch as the thread between the piston rod 42 and the nut 36.

The proximal end of nut 36 has internal threads 70 that match threads 60 of piston rod 42. The distal end of clutch 32 is configured as a dose button 72 and is permanently attached to distal end of the dose knob 31 through engagement of mechanical connectors, which can also include snap locks, an adhesive and/or a sonic weld. This connection ensures that the clutch 32 is both rotationally and axially fixed to the dose knob 31 during both dose setting and dose delivery.

In addition to thread 60 on the outer surface of the piston rod 42 and the above mentioned two longitudinal flats, the terminal proximal end has a connector, configured as a snap fit, that connects with a disc or foot 42a. At the distal end of piston rod 42 is a last dose feature of the dose setting mechanism, illustrated as an enlarged section 63. This enlarged section 63 is designed to stop the rotation of nut 36 about threads 60 when the amount of medicament remaining in the cartridge 8 is less than the next highest predetermined dose setting. In other words, if the user tries to set one of the predetermined fixed dose settings that exceeds the amount of medicament remaining in the cartridge, then the enlarged section 63 will act as a hard stop preventing the nut 36 from further rotation along threads 60 as the user attempts to reach the desired predetermined fixed dose setting

The piston rod 42 is held in a non-rotational state relative to housing 3 during both dose setting and dose delivery because it is arranged within a non-circular pass through hole in the center of piston guide 43. The piston guide 43 is both rotationally and axially fixed to housing 3. This fixation can be achieved when the piston guide 43 is a separate component from the housing 3 as illustrated in the figures or the piston guide 43 could be made integral with the housing 3. Piston guide 43 also engages the proximal end of a rotational biasing member, shown as torsion spring 90, the function of which will be explained below. This connection of the rotational biasing member 90 to the piston guide 43 anchors one end in a rotational fixed position relative to the housing 3.

The distal end of the rotational biasing member, for example torsion spring 90, is connected to the driver 41. Driver 41 is connected and rotationally fixed with the inner surface of dose sleeve 38 through a splined connection on the distal outer surface of the driver 41. On the proximal end of driver 41 on the outer surface is thread 67 that is engaged with a matching thread on the inner distal surface of the piston guide 43. The threaded connection between driver 41 and piston guide 43 has a significantly different pitch than the threaded connection between dose sleeve 38 and housing 3. The nut 36 and the driver 41 rotate together both during dose setting and dose cancellation and, as such, they perform essentially the same axial movement. However, this movement is independent from each other, i.e., the nut 36 is turned by the clutch 32 and performs an axial movement due to the thread to the piston rod 42, while the driver 41 is rotated by the dose sleeve 38 and performs an axial movement due to the thread to the piston guide 43. The driver 41 is rotating during injection also, and so it actively moves in the proximal direction during injection. But the nut 36 does not rotate during injection and as such does not perform an active axial movement. The nut 36 is only moving in the proximal direction during injection because it is being pushed axially by the driver 41. The rotating driver 41 pushing the non-rotating nut 36 causes the injection because the piston rod 42 is pushed forward due to the threaded engagement with the nut 36.

If, for example, the thread 70 of the nut 36 had a higher pitch than the thread 67 of the driver 41, the nut 36 could not freely move in the distal direction during dose setting because it would be hindered by the slower moving driver 41. As such, this would cause drug to be expelled during dose setting. Alternatively, if the thread 70 of the nut 36 had a significantly lower pitch than the thread 67 of the driver 41, the driver 41 would move away from the nut 36 during dose setting and the driver 41 would not push the nut 36 at the beginning of the injection already but would do so only after the gap is closed. Accordingly, it is preferred that the pitch of the thread 67 on the driver 41 is equal or a slightly higher than the pitch of the thread 70 on the nut 36. And, the thread 39 between the dose sleeve 38 and the housing 3 has a higher pitch than that of the nut 36 and piston rod 42. This is desirable because it yields a mechanical advantage that makes the dose delivery process easier for the user. For example, when pushing the knob 31 a distance of 15 mm, the piston rod 42 only moves by 4.1 mm. This results in a gearing ratio of about 3.6:1. A lower gearing ratio would result increase the force the user needs to complete the injection.

Because the torsion spring 90 is attached to the driver 41 and the driver 41 is rotationally fixed to the dose sleeve 38, rotation of the dose sleeve 38 in a first direction during dose setting will wind the torsion spring 90 such that it exerts a counter rotational force on the dose sleeve 38 in an opposite second direction. This counter rotational force biases the dose sleeve 38 to rotate in a dose canceling direction.

The fluid volume dispensed by an injection pen is determined by the linear translation of the threaded piston rod 42 that in turn pushes a slidable piston (bung or stopper) within the drug cartridge 8. In a number of pen-type injection devices 10, the user is able to manually adjust the desired dose setting by manipulation (e.g., turning a dose setting knob 31) of a mechanical component of the injection pen. In the case where the pen design has a dose setting knob, the knob (or a button associated with the knob) is then pushed to translate the piston rod 42 axially in a distal direction within the pen 10 to displace the drug from the cartridge 8.

The electronics module 320, either built into the pen or as an attachable and reusable separate device, is configured to interrogate the ring shaped sensors 100, 200, 210 and the optional linear sensor 400 present in the device so as to monitor and determine the above mentioned parameters relating to dose setting and dose delivery. The electronic circuit in the electronics module 320 could also include a means 312 for wireless communication using a low power protocol such as Bluetooth. The electronics can take many forms.

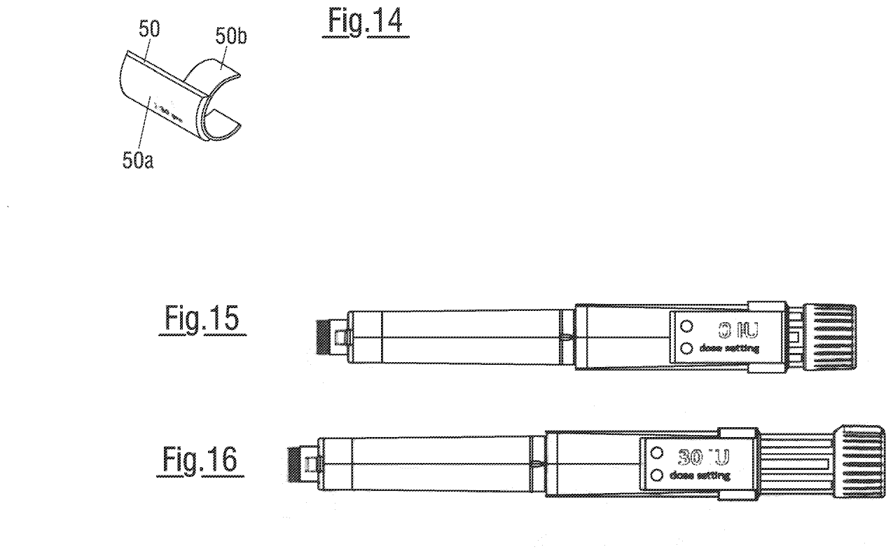

The electronics module 320 can be attached to the outside housing of the injection device, or even in some cases, can be located remotely from the injection device. One embodiment of an attachable electronics module 50 is illustrated in FIG. 14, which is preferably designed to be reusable. This electronics module 50 can be releasably attached to the injection device outer surface of housing 3 through clips and can include a display 50e to present relevant information to the user, such as, for example, the time when the last injection took place, and the dose amount of that last injection. FIG. 15 and FIG. 16 illustrate the electronics module 50 releasably attached to an injection device where the display shows a zero-dose setting (FIG. 15) and where the display shows that a dose of 30 IU has been set (FIG. 16), respectively. Clearly, other pertinent information could be displayed by the electronics module 50, such as battery charge level, temperature, alarm status, medicament identification information, connectivity status, etc. The electronics module 50 could also have one or more input features, such as buttons or touch screen features, for the user to press to activate the various features of the electronics module 50. Like the electronics module 50 shown in FIGS. 14 to 16, also the electronics module 320 shown, inter alia in FIGS. 9 to 11, can be connected to a display and can be configured to show the same information on the display as described in connection with the electronics module 50. The display can, for example, be integrated into the removable cap 306. It can also be placed remotely from the electronics module 320 and can be connectable to the electronics module 320 via a wireless connection, such as a Bluetooth or Wi-Fi connection. In this case, the display can be part of a mobile device, such as a smartphone.

The function of the complete injection device 10 and the dose setting mechanism 30 according to this disclosure will now be described. Injection device 10 is provided to a user with or without the cartridge 8 of medicament positioned within the cartridge holder 2. If the injection device 10 is configured as a reusable device, then cartridge holder 2 is connected to housing 3 of the dose setting mechanism 30 in a releasable and reusable manner. This allows the user to replace the cartridge 8 with a new full cartridge 8 when all the medicament is expelled or injected from the cartridge 8. If the device 10 is configured as a disposable injection device, then the cartridge 8 of medicament is not replaceable because the connection between the cartridge holder 2 and the housing 3 is permanent. Only through breaking or deformation of this connection can the cartridge 8 be removed from the injection device 10. Such a disposable device 10 is designed to be thrown out once the medicament has been expelled from the cartridge 8.

The user first removes the cap 1 from the device 10 and installs an appropriate pen needle 4 to the cartridge holder 2 using connector 7. If the device 10 is not pre-primed during the device assembly or does not have an automatic or forced priming feature, then the user will need to manually prime the device 10 as follows. The dose knob 31 is rotated such that a first dose stop is reached, which corresponds to a predetermined small fixed dose of medicament.

The injection device 10 of this disclosure can also have a so-called forced or automatic priming feature. Prior to using the dose setting mechanism 30, i.e., before a user could dial one of the predetermined fixed dose setting, a sliding lock would necessarily need to pushed in the proximal direction such that is moves distally relative to the dose knob 31. This axial movement forms an irreversible locking relationship between the dose knob 31 and the distal end of the clutch 32. This locking relationship also causes the dose knob 31 and clutch 32 to be rotationally fixed to each other. Before the sliding lock is engaged with the clutch 32, the clutch 32 can be rotated, which also causes rotation of the nut 36, to cause the piston rod 42 to move axially relative to the housing 3. The clutch 32 is rotated until a visual observation and/or tactile notification indicates that the foot 42a located on the piston rod 42 is in firm abutment with distal facing surface of the sliding piston 9. This abutment between the foot 42a and the sliding piston 9 will ensure that an accurate dialed dose will be delivered out of the needle cannula. The rotation of the clutch 32 is preferably performed during the assembly of the injection device 10 and likewise after ensuring abutment of the foot 42a with the sliding piston 9, the manufacturing process would cause the sliding lock to be pushed to the final, locked position.

Returning to the priming procedure, once the priming stop is reached, the user can need to cancel the priming procedure and can do so by using the dose canceling procedure. This cancellation procedure also applies to any dose setting. Dose cancellation is accomplished by turning the dose knob 31 in the opposite direction and will generate a notification that can be the same or different as the dose setting notification and/or dose delivery notification. Because the snap element 33 is rotationally fixed to the dose sleeve 38, and the dose sleeve 38 is threaded engaged to the inner surface of housing 3, rotation of the dose knob 31 during dose setting and dose cancellation causes relative rotation between the dose sleeve 38 and the housing 3. The threaded connection between the housing 3 and the dose sleeve 38 causes the dose sleeve 38, snap element 33, clutch 32, and dose knob 31 to translate axially as the dose knob 31 is rotated. During dose cancellation, these components rotate and translate axially in the opposite or proximal direction.

Rotation of the dose knob 31 also causes rotation of nut 36 about threads 60 on the outer surface of piston rod 42, which does not rotate and remains axially fixed relative to the housing 3 because of relative pitch differences in the threaded parts as explained above. The rotation of the nut 36 relative to the stationary piston rod 42, which is supported by its contact with the sliding piston 9, causes the nut 36 to translate or climb up the piston rod 42 in the distal direction. A reverse rotation during dose cancellation causes the nut 36 to translate in the reverse direction relative to piston rod 42. The distance traveled by the nut 36 to achieve the desired dose setting is directly proportional to an amount of medicament that would be expelled if the dose delivery procedure were initiated and completed. Because the pitch of the threaded connection between the dose sleeve 38 and the housing 3 is greater than pitch of the thread 70 on the nut 36, the dose sleeve 38, snap element 33, clutch 32 and dose knob 31 will travel a greater axial distance than the nut 36 as it climbs up or down the piston rod 42. The difference in axial movement would normally bind the dose setting mechanism 30 but does not do so because the difference in pitch is compensated for by the sliding splined connection between the nut 36 and the clutch 32, thus allowing the clutch 32 to travel axially a greater distance longitudinally than the nut 36. During injection, the clutch 32 pushes on the snap element 33 and as such on the dose sleeve 38. This axial force causes the dose sleeve 38 to turn due to the thread to the body housing 3. The dose sleeve 38 will only start to turn when it is pushed, if the pitch of the thread 39 is high enough. If the pitch is too low the pushing will not cause rotation because the low pitch thread 39 becomes what is called a “self-locking thread.”

Rotation of the dose knob 31 also causes rotation of the driver 41 because of the splined rotationally fixed connection to the dose sleeve 38. Since the torsion spring 90 is fixed at one end to the driver 41 and at the other end to the piston guide 43, which in turn is fixed axially and rotationally to the housing 3, the torsion spring 90 is wound up increasing in tension during dose setting. As mentioned, the torque of the torsion spring 90 exerts a counter rotational force on the dose sleeve 38. Preferably during assembly of the dose setting mechanism 30, the torsion spring 90 is pre-tensioned so that even at the zero-dose condition the torsion spring 90 exerts a counter rotational force on the dose sleeve 38. The counter rotation force provides a first fail-safe feature of the dose setting mechanism 30. This first fail-safe mechanism prevents a user from setting a dose that is not one of the finite set of predetermined dose settings. In other words, if a user is rotating the dose knob 31 such that it is between two dose stops, or between the zero dose hard stop and a first dose stop or a priming stop, and the user releases the dose knob 31, the counter rotational force of the torsion spring 90 will return the protrusion to the last engaged dose stop or to the zero dose hard stop. Additionally, during a dose cancellation procedure the counter rotational force will assist the user in rotating the dose knob 31 back down to the next lower fixed dose setting or possibly all the way back to the zero-dose setting.

During dose setting, the dose knob 31 translates out and away from the distal end of housing 3. As the dose sleeve 38 rotates and translates, the progress of the dose setting (or dose cancellation) is observed in the window 3a of housing 3 as the printed indicia 40 on the dose sleeve 38 move past the open window 3a. When a desired predetermined dose setting is reached the indicia 40 for that dose will appear in the window 3a. At this point the injection device 10 is ready for a priming procedure or, if already primed, the delivery of the medicament to an injection site. In either the case, the user will push on the dose knob 31 in the proximal direction until the zero-dose hard stop is reached and a zero-dose indicium is observed in the window 3a. During a priming step the user will observe whether medicament is expelled out of the cannula 6 of pen needle 4. If no medicament is expelled this means the piston foot 42a is not in abutment with the distal surface of sliding piston 9. The priming step is then repeated until medicament is observed exiting the cannula 6.

The dose setting mechanism 30 of the present disclosure can also have a maximum dose hard stop feature that prevents a user from setting a dose greater than the highest predetermined dose setting.

Once the dose setting mechanism 30 is primed, the user then selects and sets a desired fixed dose by repeating the same steps used for priming except that the dose knob 31 will be rotated past the priming stop until the appropriate dose stop is and the desired dose value appears in the window 3a. In some cases, it is preferred to have no indicia 40 shown in the window 3a when dialing between predetermined dose settings, while in other cases it is desirable to show indicia 40 in the window 3a that is indicative of a non-settable dose position between the fixed dose settings.

Once one of the predetermined dose settings has been dialed on the dose setting mechanism 30, the user can then exert an axial force in the proximal direction to initiate the dose delivery procedure. The axial force exerted by the user overcomes the distally directed force exerted by the second biasing member 91 causing the dose knob 31, clutch 32 and dose selector 35 to move axially in the proximal direction relative to the snap element 33 and housing 3. This initial movement rotationally fixes the clutch 32 and dose knob 31 to the housing 3 through the splined connection between the floating spline 34 and splines inside dose selector 35. The splined connection between the dose selector 35 and floating spline 34 remains engaged during dose setting and during dose delivery even though the dose selector 35 moves axially with the dose knob 31 and relative to the floating spline 34.

As the user maintains the axial force on both the dose knob 31 and the dose button 72 during the continuation of the dose delivery procedure, the clutch 32 will abut the distal end of the snap element 33 causing it to move axially in the proximal direction. The clutch 32 pushes on the snap element 33. The snap element 33 is fixed to the dose sleeve 38, so the clutch 32 pushes on the dose sleeve 38. As the dose sleeve 38 has a thread 39 with a sufficiently high pitch relative to the body 3, the axial force on the dose sleeve 38 will cause the dose sleeve 38 and as such the snap element 33 to turn relative to the body 3, and by turning relative to the body 3 it moves in the proximal direction. The dose selector 35 slides into the housing 3 but does not rotate relative to the housing 3 due to the splined engagement with the housing 3. The rotation of the dose sleeve 38 also causes rotation of the driver 41 into the threaded connection with piston guide 43, which drives the piston rod 42 proximally and results in a concurrent detensioning of torsion spring 90. The driver 41 does not directly drive the piston rod 42. As the driver 41 rotates, the driver 41 moves in the proximal direction and pushes the nut 36 forwards. As the nut 36 doesn't turn, the driver 41 pushes the nut 36 and the piston rod 42 forward.

The nut 36 does not rotate during dose delivery because of the rotationally fixed relationship with clutch 32 that is rotationally fixed to the housing 3 through rotationally fixed relationship of the dose knob 31, floating spline 34 and the housing 3. The nut 36 therefore can only move axially carrying the piston rod 42 with it because the piston rod 42 is prevented from rotating by the non-circular opening engaged with flats on the piston rod 42. The piston rod 42 is moved axially the same distance that the nut 36 originally translated relative to the piston rod 42 during dose setting. This axial movement without rotation is caused by the rotational and axial movement of the proximal end of the driver 41 in abutment with a flange 36a on nut 36. Axial movement of the piston rod 42 causes the sliding piston 9 to also move axially relative to the inside walls of the stationary cartridge 8 forcing an amount of medicament out of the needle cannula 6 that is equivalent to the predetermined fixed dose that was set during the dose setting procedure.

If the user stops or halts the dose delivery procedure by removing the axial force on the dose knob 31 a fail-safe mechanism is activated. Removal of the axial force causes the compression spring 91 to bias the dose knob 31 in the distal direction. If the user halts the dose delivery between two predetermined fixed dose settings, then the dose knob 31 and the axially fixed dose selector 35 will both be prevented from moving proximally because of a projecting rib inside the dose selector 35 that will stop the axially movement of dose selector 35 and dose knob 31. Without this projecting rib, the dose selector 35 would move distally such that the dose knob 31 would re-engage with the snap element 33, thus placing the dose knob 31, clutch 32 and nut 36 back into rotational engagement with the snap element 33. The torque exerted on the snap element 33 through the driver 41 would then counter rotate the nut 36, thus reducing the set dose by an unknown amount. This counter rotation would continue until the next lowest predetermined fixed dose setting is reached, where the corresponding dose stop would stop the counter rotation. Therefore, a resumption of the halted dose delivery procedure will continue without an unknown decrease in the set dose, thus allowing the originally set predetermined dose to be delivered. A halted dose delivery could be determined using the linear sensor 400 described above because the electronics module 320 would sense a rate change of movement or time lag during dose delivery. Likewise, a halted dose delivery could be determined and recorded by using a clock function of the electronics module 320 that would sense no movement of the linear sensor 400 over a period of time for the injection corresponding to the halted injection.

As shown in FIGS. 4 and 12, the drug delivery devices 10, 600 described in connection with the previous Figures each have a sensor mechanism that comprises the electronics module 320 and a dose setting sensor 700. The dose setting sensor 700 is configured as a rotation sensor. It senses the rotation of the knob 31, 631 during dose setting and generates sensor signals that are indicative of the sensed rotation. The dose setting sensor 700 comprises an active sensing circuit 706 and a passive dose setting encoder 707. The sensing circuit 706 is positioned on the electronics module 320 and can be configured as a separate electronic component, such as an integrated circuit, or it can be part of the microcontroller 310.

The sensing circuit 706 is connected to the dose setting encoder 707 of the dose setting sensor 700 via electrical conductors 701, 702, 703, such as the leads 101, 102, 103. The sensing circuit 706 is configured to monitor a state of the dose setting encoder 707 and to generate sensor signals, such as the pulsed signals A, B shown in FIG. 8, that are indicative of the monitored state and corresponding state changes. As such, the dose setting encoder 707 is configured as an incremental rotary encoder that generates the signals A, B in quadrature. The pulses of the signals A, B are thereby created at a rate that is proportional to the angular velocity of the rotation of the knob 31, 631. The sensing circuit 706 determines the direction of rotation and the position of the knob 31, 631 from the pulses of the signals A, B.

The microcontroller 310 constitutes a logic unit and is configured to evaluate the sensor signals received from the sensing circuit 706 of the dose setting sensor 700. Thereby, the microcontroller 310 is configured to determine both an amount and a direction of the rotation of the knob 31, 631 during dose setting. The microcontroller 310 then deduces the actually set dose from this information.

The sensor mechanism shown in FIGS. 4 and 12 further comprises a dose delivery sensor 800 with a dose delivery encoder 807 and a sensing circuit 806. The dose delivery sensor 800 is configured to sense the position of an element of the injection device 10, 600 that moves axially during dose delivery, such as, for example, the sleeve 35, 635. The dose delivery sensor 800 can be configured as a linear sensor that is configured to monitor the axially moving element during the entire process of dose delivery. Such a linear sensor, can, for example, be the linear sensor 400 shown in FIGS. 12 and 13, the dose delivery encoder 807 of which comprises the contacts 420 on the sleeve 635 and the static contact 405 within the housing 3. The dose delivery encoder 807 is part of a sensor portion of the dose delivery sensor 800 that is rotationally fixed with respect to the housing 3.

The dose delivery sensor 800 can also be configured as a switch, such as the end of dose switch 500, that only changes state when the axially moving element reaches a predetermined axial position, such as the end of dose position after complete injection of the dose set by the knob 631. A switching part or electrical connector of such a switch, like the switching part formed by the parts 500a, 500b of the end-of dose sensor 500, can be configured as a sensor portion of the dose delivery sensor 800 that is rotationally fixed with respect to the housing 3.

The dose delivery encoder 807 of the dose delivery sensor 800 is connected to the sensing circuit 806 via electrical conductors 704, 705. Like the sensing circuit 706 of the dose setting sensor 700, the sensing circuit 806 of the dose delivery sensor 800 is configured to monitor the state of the dose delivery encoder 807 and to generate sensor signals that are indicative of the monitored state and corresponding state changes.

A first embodiment of a dose setting sensor 700 according to the present invention is shown in FIGS. 5 to 7 and 9 to 11, a second embodiment is shown in FIGS. 18A, 18B and 19. The dose setting sensors 700 are configured as rotary sensors that have dose setting encoders 707 that comprise at least one first sensor element 710, such as the rotating ring sensor 100 of the first embodiment, that is rotationally fixed with respect to the knob 31, 631 during dose setting and at least one second sensor element 720, such as the stationary ring sensors 200, 210, that is rotationally fixed with respect to the housing 3 during dose setting. Rotation of the knob 31, 631 relative to the housing 3 during dose setting then causes the first sensor element 710 to rotate with respect to the at least one second sensor element 720.

With both embodiments of the dose setting sensor 700, the electronics module 320 and both the first and second sensor elements 710, 720 are axially fixed with respect to each other. Additionally, they are axially fixed with respect to the knob 31, 631, for example via the ring aligner 300. Furthermore, they are axially movable with respect to the housing 3 and move together with the sleeve 35, 635 during dose setting.

When being incorporated into the further injection device 600, the electronics module 320 and both the first and second sensor elements 710, 720 are axially movable with respect to the sleeve 635 to allow the knob 631 to be displaced from the dose setting position 654 into the dose delivery position 655. To simultaneously achieve rotational fixation and axial movement between the housing 3 and the second sensor elements 720, the second sensor elements 720 are slideably connected to the housing 3 via a keyed connection that allows for axial movement and inhibits rotational movement of the second sensor elements 720 with respect to the housing 3. The keyed connection comprises at least one lug, such as lugs 203 on the outer circumferential surface of the second sensor elements 720 shown in FIG. 6, that is slideably received in a longitudinal recess. The longitudinal recess is orientated parallel to the axial direction and is configured in a component of the injection device 600 that is rotationally fixed with respect to the housing 3.

With the further injection device 600, the component is an inset 640 that is axially and rotationally fixed with respect to the sleeve 635 and that is received in the sleeve 635 at its distal end in the section having the projection 636. The inset 640 has a hollow cylindrical body that has longitudinal recesses 642 at its inner lateral surface, see FIGS. 7, 10 and 19. Other embodiments of the further injection device 600 can lack the inset 640 and the recesses 642 can then be configured at an inner lateral surface of the sleeve 635 itself.

As can be seen from FIGS. 5A and 5B, the contact points 106, 107, 108, 109 of the first embodiment of the dose setting sensor 700 form contact elements 114 that are configured as two-dimensional surface contacts. With the embodiment shown in FIGS. 5A and 5B, the contact elements 114 are located at end faces 104, 105 of a ring-like cylindrical insulating carrier. The insulating carrier is configured as a rigid structure, such as a printed circuit board, and thus also constitutes a rigid support member of the contact elements 114. The first and third contact point 106, 108, which are both electrically connected to the first conductor 701, constitute contact elements 114 of a first contact structure 711 of the first sensor element 710, the second contact point 107 constitutes a contact element 114 of a second contact structure 712 of the first sensor element 710 and the fourth contact point 109 constitutes a contact element 114 of a third contact structure 713 of the first sensor element 710.

As far as no differences are described or apparent from the Figures, the second embodiment of the dose setting sensor 700, which is shown, inter alia, in FIGS. 18 to 19, is configured as it is disclosed in connection with the first embodiment shown in FIGS. 5 to 7 and 9 to 11 and vice versa.

As can be seen from FIG. 19, which semi-transparently depicts the sleeve 635 and the inset 640 of the further injection device 600, the second embodiment of the dose setting sensor 700 has a first sensor element 710 that comprises contact elements that are arranged next to each other along a circumferential direction around the longitudinal axis 652. The second sensor element 720 is configured as an electrically conducting metal structure that has a metal ring 721, which surrounds the longitudinal axis 652 and holds electrically conducting and spring-loaded linking elements 725, 736.

The linking elements 725, 726 are configured to contact the contact elements of the first sensor element 710 in the radial direction perpendicular to the longitudinal axis 652. They intermittently electrically contact the contact elements of the first sensor element 710 upon rotation of the knob 631 and the first sensor element 710 of the dose setting sensor 700 during dose setting. Like the second sensor elements 720 of the first embodiment of the dose setting sensor 700, also the second sensor element 720 of the second embodiment of the dose setting sensor 700 has radially projecting lugs 728 that are slideably received in the recesses 642 of the inset 640 of the sleeve 635 and prevent the second sensor element 720 from rotating during dose setting.

The sensor mechanism shown in FIG. 19 also comprises an electrical connector 820 that is configured to electrically conductively connect the first part 500a of the end of dose switch 500, which is rotationally fixed with respect to the housing, to the electronics module 320 when the knob 631 is proximally moved from the dose setting position 654 into the dose delivery position 655. This proximal movement transfers the electrical connector 820 from an open state into a closed state.

The electrical connector 820 comprises a first part 822 that is electrically conductively connected to the electronics module 320 and that is rotationally and axially fixed with respect to the knob 631. Therefore, the first part 822 rotates together with the knob 631 during dose setting. The first part 822 of the connector 820 is configured as a circumferential arrangement 825 of surface contacts 826, 827 distributed around the longitudinal axis 652 in the circumferential direction. Thereby, first contacts 826 that are electrically conductively connected with each other and second contacts 827 that are electrically conductively connected with each other and electrically isolated from the first contacts 826 are alternately placed next to each other around the longitudinal axis 652.

The electrical connector 820 further comprises a second part 830 that is electrically conductively connected to the first part 500a of the dose delivery sensor 500 and that is rotationally and axially fixed with respect to the sleeve 635 and the housing 3. The second part 830 is configured to engage with the first part 822 of the connector 820 when the knob 631 is moved from the dose setting position 654 into the dose delivery position 655 and the electrical connector 820 is transferred in the closed state. Furthermore, the second part 830 is configured to disengage from the first part 822 when the knob 631 is moved back into the dose setting position 654 and the electrical connector 820 is transferred back into its open state.

The second part 830 of the electrical connector 820 is configured as a metal structure that has a spring-loaded connector contact 832. The connector contact 832 is configured to bear against the first and second contacts 826, 837 of the first part 822 of the electrical connector 820 in the radial direction perpendicular to the longitudinal axis 652. It comprises a first contact element 833 and a second contact element 834, whereby the first and second contact element 833, 834 are electrically isolated from each other and configured to electrically contact neighboring pairs of the first and second contacts 826, 827 of the first part 822 of the connector 820.

The knob 631 is only movable into the dose delivery position 655 if it is located in well-defined and discrete rotational positions that correspond to distinct settable doses. With the further injection device 600, these rotational positions are defined by longitudinal grooves 641 within the inner lateral surface of the inset 640. The grooves 641 are circumferentially distributed around the longitudinal axis 652 and receive the couplings 31a of the knob 631 when the knob 631 is moved in the proximal direction. The angular distances between the individual grooves 641 then determine the rotational positions, in which the knob 631 is allowed to move into the dose delivery position 655.

The first and second contacts 826, 827 of the first part 822 of the connector 820 are distributed around the longitudinal axis 652 in a way that, for each of the rotational positions of the knob 631, the second part 830 of the connector 820 contacts a distinct pair of the first and second contacts 826, 827. For the further injection device 600, which has twenty longitudinal grooves 641 and therefore twenty settable dosing positions per revolution of the knob 631, the first part 822 therefore comprises twenty pairs of the first and second contacts 826, 827, one for each settable dosing position per revolution.

With alternative embodiments of the electrical connector 820, the first and second contacts 826, 827 of the first part 822 can be sized and positioned in a way that the first contact element 833 of the second part 830 alternately contacts one of the first contacts 826 and one of the second contacts 827 when the knob 631 is consecutively moved through neighboring dosing positions, while the second contact element 834 alternately contacts the corresponding other one of the neighboring first and the second contacts 826, 827. With such an embodiment, the circuit components that are connected to the electronics module 320 via the electrical connector 820 can be configured in a way that it does not matter whether the first contact element 833 is connected to the first contacts 826 and the second contact element 834 is connected to the second contacts 827 or vice versa. Therefore, such an embodiment of the electrical connector 820 can, for example, connect the end of dose sensor 500 shown in FIG. 18 to 19 or the linear sensor 400 shown in FIGS. 12 and 13 to the electronic module 320. With the further injection device 600, which has twenty longitudinal grooves 641, such an embodiment of the electrical connector 820 can then have twelve first contacts 826 and twelve second contacts 827 that are alternately placed next to each other in the circumferential direction.

The first part 500a of the end of dose switch 500 comprises two electrically isolated switching contacts, each of which is electrically connected to a separate one of the first and second contact elements 833, 834 of the second part 830 of the electrical connector 820. The switching contacts are electrically connected with each other by the second part 500b of the end of dose switch 500 as soon as the sleeve 635 abuts against the housing 3 at the end of the delivery of the set dose. Therefore, the first and second part 500a, 550b form a further electrical connector and the end of dose switch 500 is actuated when this further electrical connector is transferred from its open state into its closed state at the end of the delivery of the set dose.

The second part 500b of the end of dose switch 500 is configured as a single metal piece, which is co-molded into the housing 3. Likewise, the connector 820 with the first part 500a of the end of dose switch 500 is also configured as a metal member, which is co-molded into the sleeve 635 and comprises two electrically isolated metal pieces. Thereby, first portions of the metal pieces, which form the first part 822 of the electrical connector 820, are exposed at the inner lateral surface of the inset 640 and second portions of the metal pieces, which comprise the first part 500a of the end of dose switch 500 are exposed at the outer surface of the shell portion 637 of the sleeve 635.

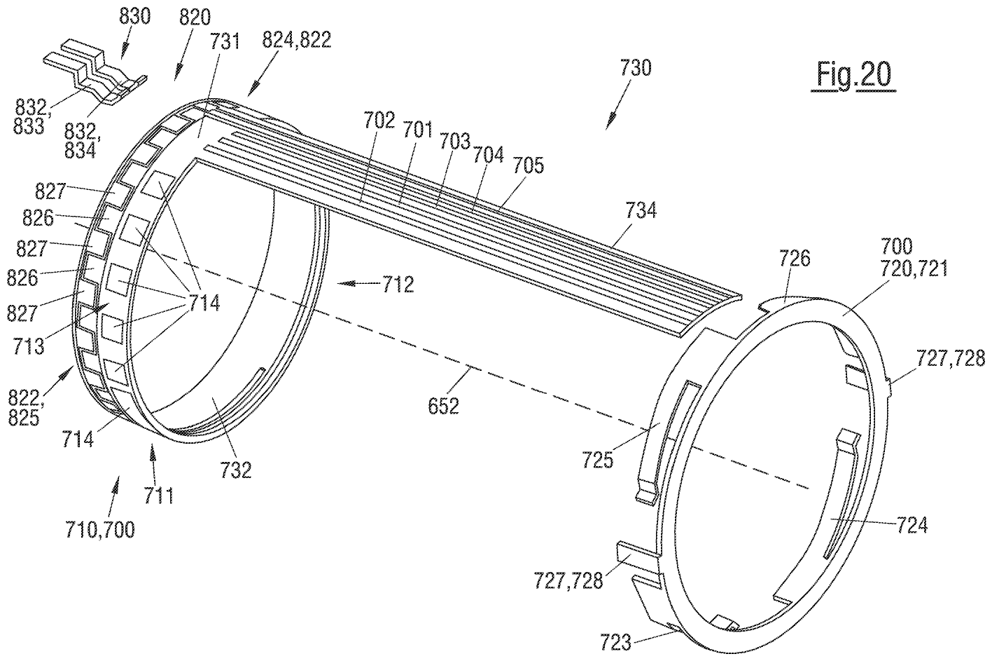

FIG. 20 depicts an exploded view of the dose setting sensor 700 and the electrical connector 820 shown in FIG. 19. The contact elements 714 of the first sensor element 710 of the dose setting sensor 700 and the first and second contacts 826, 827 of the first part 822 of the electrical connector 820 are circumferentially distributed on a cylindrical outer lateral surface 731 of a ring-shaped section 732 of an electrically insulating carrier 730. Thereby, the contact elements 714 and the first and second contacts 826, 827 are configured as two-dimensional surface contacts.

The carrier 730 further comprises a longitudinal section 734 that is orientated parallel to the longitudinal axis 652 and that carries the first, second and third conductor 701, 702, 703, which electrically conductively connect the first part 710 of the dose setting sensor 700 to the electronics module 320, and the fourth and fifth conductor 704, 705 that electrically connect the first part 822 of the electrical connector 820 to the electronics module 320. In the embodiment shown in FIG. 20, the carrier 730 is configured as a flexible printed circuit board.

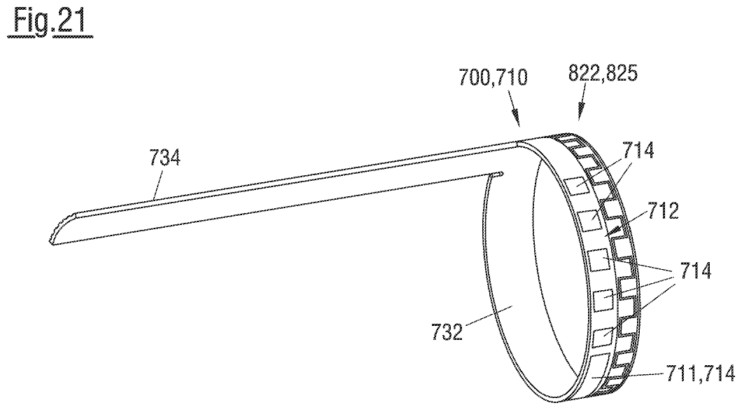

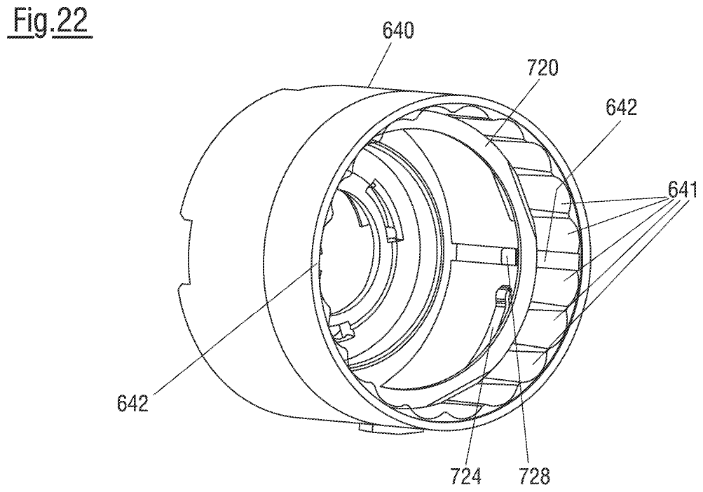

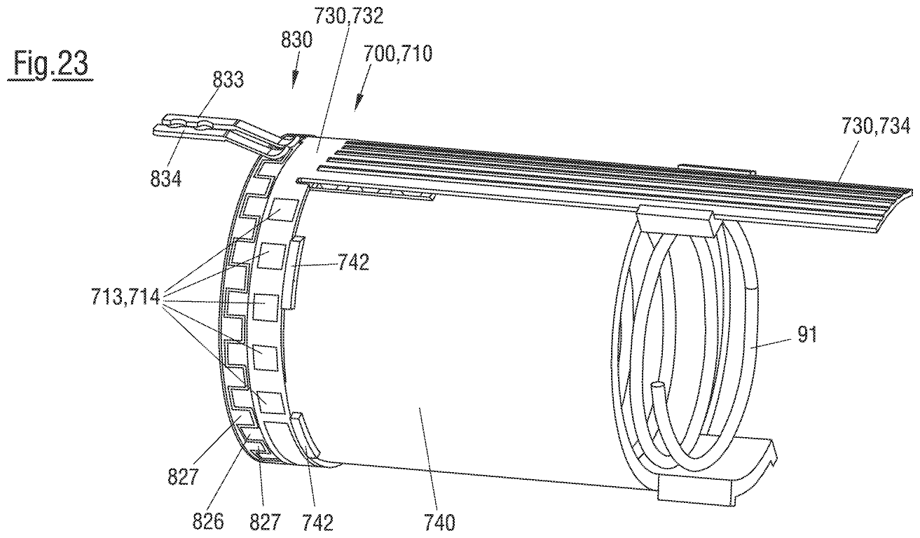

The first sensor element 710 of the dose setting sensor 700 comprises a first contact structure 711 that is electrically conductively connected to the first conductor 701, a second contact structure 712 that is electrically conductively connected to the second conductor 702 and a third contact structure 713 that is electrically conductively connected to the third conductor 703. The first contact structure 711 comprises a single contact element 714 that is elongated in the circumferential direction. The third contact structure 713 comprises five contact elements 714 that are positioned at a distance from each other along the circumferential direction at one side of the contact element 714 of the first contact structure 711. The second contact structure 712, which is depicted in FIG. 21, also comprises five contact elements 714 that are positioned at a distance from each other along the circumferential direction at the other side of the contact element 714 of the first contact structure 711. Each contact structure 711, 712, 713 covers essentially a fourth of the circumference of the lateral surface 731 of the carrier 730. With other embodiments, each contact structure 711, 712, 730 can also cover essentially a third of the circumference of the lateral surface 731 of the carrier 730.

The second sensor element 720 of the dose setting sensor 700 is configured as a punched and bent sheet metal. It has a support ring 721 that carries a first linking element 723, a second linking element 724, a third linking element 725 and a fourth linking element 726. The linking elements 723, 724, 725, 726 are configured as spring-loaded elements that radially bear against the cylindrical lateral surface 731 of the ring-shaped section 732 that carries the contact elements 714 of the first sensor element 710.

The four linking elements 723, 724, 725, 726 are arranged in pairs opposite each other, with the two pairs being rotated by 90° with respect to each other. This balances the forces that are exerted by the individual linking elements 723, 724, 725, 726 on the first sensor element 720. On its outer side, the second sensor element 720 has the radially projecting lugs 728 that, together with the recesses 642 at the inner lateral surface of the inset 640, form the keyed connection that prevents the second sensor element 720 from rotating during dose setting. This keyed connection is also shown in FIG. 22 and works in the same way as the keyed connection between the lugs 203 of the second sensor elements 200, 210 and the recesses 642 of the inset 640 shown FIGS. 7 and 10.

As can be seen from FIG. 20, the first contacts 826 of the first part 822 of the electrical connector 820 are connected via the fourth conductor 704 to the electronics module 320 and the second contacts 827 of the first part 822 of the electrical connector 820 are connected via the fifth conductor 705 to the electronics module 320. The first contacts 826 are thereby connected to each other and to the fourth conductor 704 via an electrically conducting connection in the circumferential direction that is located distally from the first and second contacts 826, 827. The second contacts 827 are likewise connected to each other and to the fifth conductor 705 via an electrically conducting connection in the circumferential direction that is located proximally from the first and second contacts 826, 827.

With the embodiment shown in FIG. 20, the cylindrical lateral surface 731 that carries the contact elements 714 of the first sensor element 710 and the contacts 826, 827 of the first part 822 of the electrical connector 820 is an outer surface of a cylindrical member of the injection device 600. In alternative embodiments, the cylindrical lateral surface 731 can also be an inner surface of a cylindrical member of the injection device 600 that is rotationally fixed with respect to the knob 631. With these embodiments, the second sensor elements 720 can be placed within the cylindrical member and the linking elements 723, 724, 725, 726 can radially bear against the cylindrical lateral surface 731 in the outward direction. The lugs 728 of the keyed connection that prevents the second sensor element from rotating can then project from the ring 721 in the inward direction and can be guided in a member of the injection device 600 that is positioned within the ring 721 of the second sensor element 720 and that is rotationally fixed with respect to the housing 3. Furthermore, the connector contact 832 of the electrical connector 820 can then also radially bear against the cylindrical surface 731 in the outward direction when the electrical connector 820 is in its closed state.

FIG. 23 depicts the carrier 730 mounted on a support member 740. The support member 740 is a rigid element of the injection device 600. It has a lateral cylindrical surface on which the carrier 730 is placed. With the embodiment shown in FIG. 23, this surface is the outer lateral surface of the support member 740. The support member 740 is configured as a hollow cylindrical structure and accommodates in its inside the second biasing member 91 that biases the knob 631 in the distal direction.

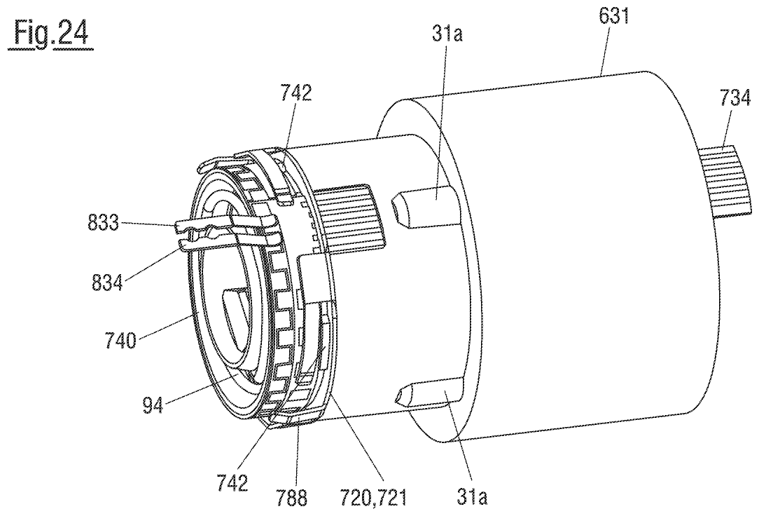

Next to and distally from the ring-shaped section 732 of the carrier 730, the support member 740 has radial protrusions 742. As can be seen from FIG. 24, which shows the carrier 730 and the second sensor element 720 mounted to the knob 631, these protrusions 742 hold the second sensor element 720 against the proximal end of the knob 631, whereby the ring 721 of the second sensor element 720 is positioned between the protrusions 742 and the knob 631. Thereby, the second sensor element 720 is axially fixed with respect to the knob 631 and the carrier 730.

The support member 740 is mounted rotationally and axially fixed to the knob 631 so that also the carrier 730 with the first sensor element 710 of the dose setting sensor 720 and the first part 822 of the electrical connector 820 is rotationally and axially fixed with respect to the knob 631.

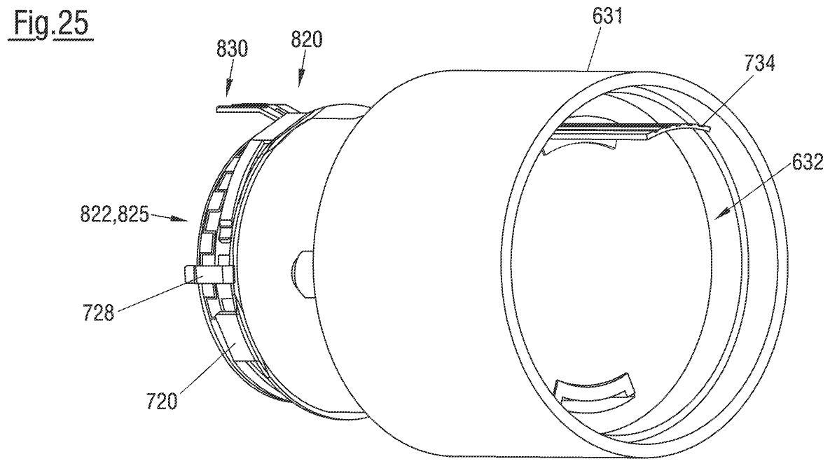

As can be seen from FIG. 25, the longitudinal section 734 of the carrier 730 protrudes into a cavity 632 of the knob 631 that is open at the distal end of the knob 631. This cavity 632 accommodates the electronics module 320. With the electronics module 320 mounted inside the cavity 632, the conductors 701, 702, 703, 704, 705 on the longitudinal section 734 of the carrier 730 are electrically conductively connected to the electronics module 320 via a releasable electric connector. Thereby, the distal end of the longitudinal section 734 of the carrier 730 forms a connection element that is received by a connector part placed on the proximal side of the electronics module 320.

With alternative embodiments of the injection device 600, the support member 740 can also function as the carrier 730. The cylindrical surface 731, on which the conductors 701, 702, 703, 704, 705 and the contact elements 714, as well as the first and second contacts 826, 827 of the electrical connector 820 are placed, is then formed by the cylindrical lateral surface of the support member 740. With these embodiments, the conductors 701, 702, 703, 704, 705, the contact elements 714, and the first and second contacts 826, 827, as well as all other conducting structures carried by the support member 740 can be co-molded into the support member 740 and can be exposed at the outer surface of the support member 740. In these cases, the support member 740 can also have a longitudinal section that protrudes, like the longitudinal section 734 of the carrier 730 shown in FIG. 25, into the cavity 632 at the lateral end of the knob 631 and is received by the connector part of the electronics module 320.

Both the first embodiment of the dose setting sensor 700 shown in FIGS. 5 to 7 and 9 to 11 and the second embodiment of the dose setting sensor 700 shown in FIGS. 18 to 25 have dose setting encoders 707 that generate sensor signals that are indicative of the direction of rotation of the knob 631.

With the embodiments, these sensor signals are the signals A and B, which are shown in FIG. 8 upon rotation of the knob 631 with constant angular velocity. The sensor signals A, B are pulsed electrical signals that are generated in quadrature. Sensor signal A is generated by opening and closing the electrical connection between the first contact structure 711 and the second contact structure 712 via the rotating second sensor element 200, 720 and sensor signal B is generated by opening and closing the electrical connection between the first contact structure 711 and the third contact structure 713 via the rotating second sensor element 210, 720.

When rotating the knob 631 into a given direction, the signal pulses of signal B are shifted with respect to the signal pulses of signal A by a quarter of a pulse period. To determine the direction of rotation of the knob 631, the electronics module 320 is configured to trigger on a given edge, such as the rising or falling edge, of one of the signals A, B and to monitor the state of the other one of the signals A, B upon triggering. This state differs depending on whether the knob 631 rotates clockwise or counterclockwise. If for example, the electronics module 320 triggers on the rising edge of signal B shown in FIG. 8, signal A would be in the OFF state upon triggering during clockwise rotation and in the ON state upon triggering during counterclockwise rotation of the knob 631.

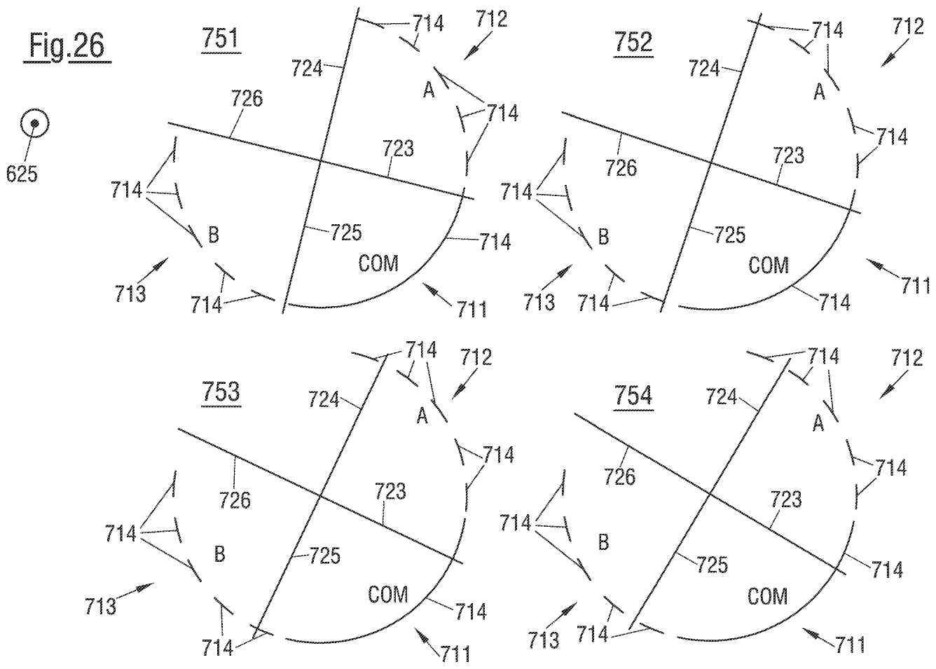

FIG. 26 schematically depicts the generation of the sensor signals A, B by the dose setting encoder 707 of the second embodiment of the dose setting sensor 700. FIG. 26 thereby shows the arrangement of the contact elements 714 of the first sensor element 710 in the circumferential direction around the longitudinal axis 652. Furthermore, FIG. 26 shows the position of the linking elements 723, 724, 725, 726 of the second sensor element 720 relative to the contact elements 714 of the first sensor element 710 upon rotation of the knob 631 in the clockwise direction.

In FIG. 26 the second sensor element 720 is depicted in a first relative position 751, a subsequent second relative position 752, a subsequent third relative position 753 and a subsequent fourth relative position 754 with respect to the first sensor element 710. In each of the positions 751, 752, 753, 754, the first linking element 723 contacts the contact element 714 of the first contact structure 711.

In the first relative position 751 the second linking element 724 makes electrical contact with a first one of the contact elements 714 of the second contact structure 712. This electrically connects the second contact structure 712, which is electrically connected to the second conductor 702, to the first contact structure 711 and the first conductor 701. The sensing circuit 706 then senses a common signal applied to the first conductor 701 via the second conductor 702, which in turn generates a rising edge of signal A from the ON state into the OFF state. Simultaneously, the third linking element 725 is positioned at an insulating area in between the first contact structure 711 and the third contact structure 713 and the fourth linking element 726 is positioned at an insulating area in between the third contact structure 713 and the second contact structure 712. Therefore, the sensing circuit 706 does not sense the common signal via the third conductor 703 connected to the third contact structure 713, which corresponds to the ON state of signal B.

In the subsequent second relative position 752, the second linking element 724 is still electrically connected to the second contact structure 712, while the third linking element 725 makes electrical contact with a first one of the contact elements 714 of the third contact structure 713. This generates a rising edge of signal B from the ON state to the OFF state while signal A is in the OFF state.

In the subsequent third relative position 753, the second linking element 724 has moved out of electrical contact with the second contact structure 712, while the third linking element 725 still makes electrical contact with the first one of the contact elements 714 of the third contact structure 713. This has generated a falling edge of signal A from the OFF state to the ON state while signal B is in the OFF state. Furthermore, in the subsequent fourth relative position 754, the third linking element 725 has moved out of contact with the third contact structure 713, while the second linking element 724 stayed out of electrical contact with the second contact structure 712. This has generated a falling edge of signal B from the OFF state to the ON state while signal A is in the ON state.

When completing a quarter of a rotation from the first relative position 751, the first linking element 723 moves out of contact with the first contacting structure 711 and starts to contact the third contacting structure 713, while the second linking element 724 moves out of contact with the second contacting structure 712 and starts to contact the first contacting structure 711. Simultaneously, the third linking element 725 moves out of contact with the third contacting structure 713 and the fourth linking element 726 moves into contact with the second contacting structure 712.

In connection with the previous Figures, the electrical connector 820 for connecting the rotationally fixed sensor portion of the dose delivery sensor to the electronics module 320 has been described for a dose delivery sensor that comprises the end of those switch 500. The electrical connector 820 can also connect a rotationally fixed sensor portion of the dose delivery sensor to the electronics module 320 that is configured as the linear encoder of the linear sensor 400 shown in FIGS. 12 and 13.

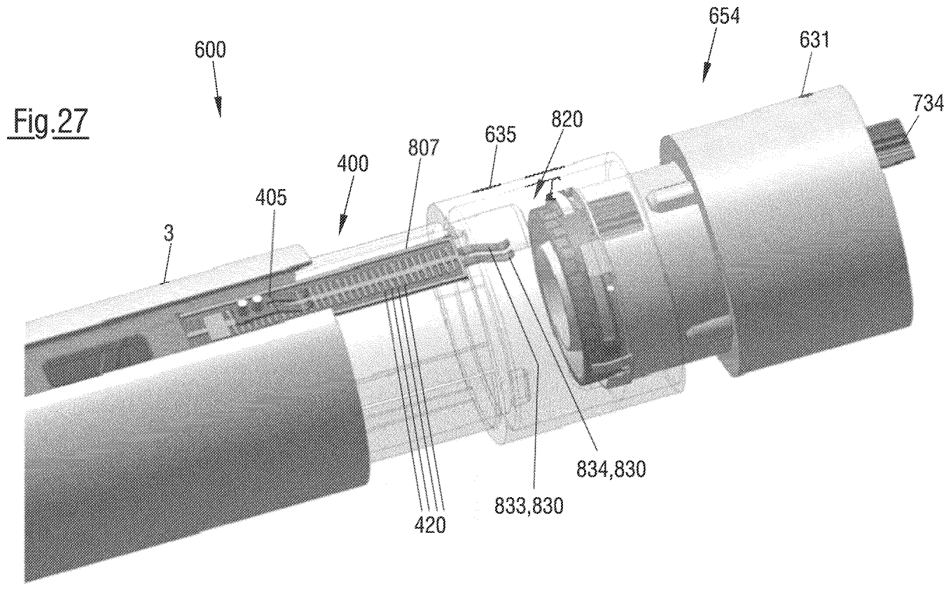

Such a configuration is depicted in FIG. 27. The dose delivery encoder 807, which forms the rotationally fixed sensor portion of the dose delivery sensor, is positioned on the outer surface of the sleeve 635. It is electrically connected to the second part 830 of the electrical connector 820. For each pair of opposing contacts 420, one of the contacts 420 is electrically connected to the first contact element 833 and the other one of the contacts 420 is electrically connected to the second contact element 834 of the second part 830 of the electrical connector 820.

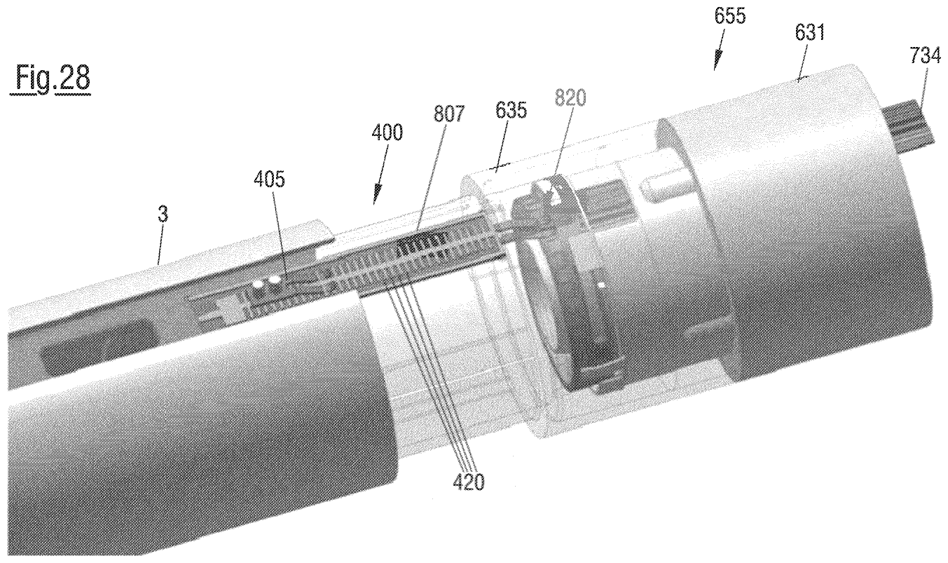

FIG. 27 shows the knob 631 in the distal dose setting position 654, in which the electrical connector 820, which comprises the first and second contact element 833, 834 in combination with the first and second contacts 826, 827, is in its open state. FIG. 28 shows the knob 631 after movement into the proximal dose delivery position 655, in which the electrical connector 820 is in its closed state. Upon linear movement of the sleeve 635 in the proximal direction during dose delivery, the static electric contact 405 sequentially closes electrical contact between subsequent pairs of opposing contacts 420. This generates a pulsed sensor signal that is detected by the electronics module 320.

Embodiments of the injection devices 10, 600 can have sensor mechanisms that comprise one of the embodiments of the rotary dose setting sensor 700 and the end of dose switch 500. A method 900 for operating such devices is shown in FIG. 2. The method 900 comprises monitoring the state of the dose setting sensor 700. Upon rotation of the knob 631 by the user (901), the electronics module 320 wakes up (911) from idle mode and senses (912) via the dose setting sensor 700 a setting of a dose if the knob 631 is rotated in one direction and a possible cancellation of a dose if the knob 631 is rotated in the opposite direction. As soon as the user has set (903) a desired dose and the electronics module 320 detects a stop of rotation of the knob 631, the electronics module transitions into idle mode (913) to save energy.

As soon as the knob 631 is pushed in the proximal direction to start delivery of the set dose, the electronics module 320 wakes up from idle mode again (911) and stores (914) the dialed dose together with a timestamp permanently in a non-volatile memory of the electronics module 320.

Then the user injects (905) the set dose and, upon finishing (906) the injection, the electronics module detects (915) activation of the end of dose switch 500. The electronics module 320 then stores (916) an information about the completion of the injection together with a timestamp in the non-volatile memory. Finally, the electronics module 320 returns into idle mode (913).

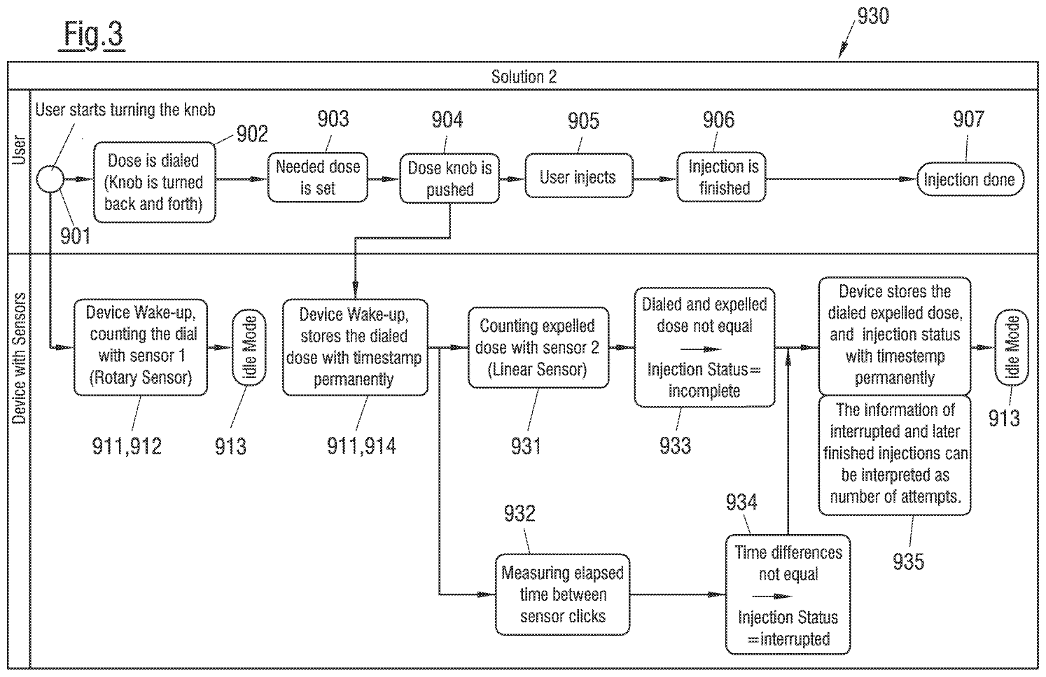

Other embodiments of the injection devices 10, 600 can have sensor mechanisms that comprise one of the embodiments of the rotary dose setting sensor 700 and a linear dose delivery sensor, such as the linear dose delivery sensor 400. A method 920 for operating such devices is shown in FIG. 3.

Like the method 900 shown in FIG. 2, the method 920 starts with the electronics module 320 being in the idle state and monitoring the state of the rotational dose setting sensor 700. The method 920 also comprises the waking up 911 from the idle mode, the sensing 912 of the setting of a dose and the transition 913 into the idle mode, once a dose has been set. After the waking up 911 of the electronics device 320 and the storing 914 of the set dose, the method 920 comprises a sensing 931 of the expelled dose by monitoring the linear sensor 400 with the electronics device 320. Furthermore, the method 920 comprises measuring time differences that elapse during the individual sensor pulses generated by the linear sensor 932 and determining a speed of injection from the time intervals (932).

The method 920 further comprises comparing 933 of the injected dose determined from the axial movement sensed by the dose delivery sensor 400 with the set dose determined from the rotational movement sensed by the dose setting sensor 700. If the injected dose differs from the set dose, the electronics module 320 records an only partial or incomplete delivery of the set dose.

Furthermore, the method 920 comprises comparing 934 the individual time differences measured between the individual sensor pulses of the linear sensor 400 with each other. If the individual time differences differ, for example by more than a predetermined threshold, the electronics module 320 sets an information about an interrupted injection.

The method 920 then comprises storing 935 the set dose, the injected dose and the information on a possibly interrupted injection together with a timestamp in the non-volatile memory. Finally, the electronics module 320 transitions into the idle mode again (913).

The above-presented description and figures are intended by way of example only and are not intended to limit the present invention in any way except as set forth in the following claims. It is particularly noted that persons skilled in the art can readily combine the various technical aspects of the various elements of the various exemplary embodiments that have been described above in numerous other ways, all of which are considered to be within the scope of the invention.

While the invention herein disclosed has been described by means of specific embodiments and applications thereof, numerous modifications and variations can be made thereto by those skilled in the art.

1. A delivery device comprising:
a housing configured to receive a cartridge for a drug, the housing having a longitudinal extent along a longitudinal axis;
a controller configured to set a dose to be delivered by the delivery device; and
a sensor mechanism configured to record the dose set by the control element,
the controller configured to rotate with respect to the housing around the longitudinal axis during dose setting and to axially move with respect to the housing along the longitudinal axis during dose setting,
the sensor mechanism comprising an electronics module and a dose setting sensor configured to sense the rotation of the control element during dose setting,
the dose setting sensor comprises a first sensor element and a second sensor element,
the first sensor element having contact elements electrically connected to the electronics module via electrical conductors,
the electronics module and the first sensor element rotationally and axially fixed with respect to the controller,
the second sensor element rotationally fixed with respect to the housing and rotationally movable with respect to the controller,
the first sensor element and the second sensor element at least temporarily positioned outside of the housing along the longitudinal axis during dose setting.

2. The delivery device according to claim 1,
wherein the second sensor element is axially fixed with respect to the controller.

3. The delivery device according to claim 1,
wherein the controller is connected to the housing via a connector,
the connector is axially movable and rotationally fixed with respect to the housing during both dose setting and dose delivery,
the controller is rotationally movable with respect to the connector during dose setting,
wherein the second sensor element is rotationally fixed with respect to the connector.

4. The delivery device according to claim 3,
wherein the controller is axially movable with respect to the connector from a dose setting position into a dose delivery position, and
the controller and the first sensor element are rotationally fixed with respect to the connector in the dose delivery position.

5. The delivery device according to claim 1,
wherein the second sensor element is axially movable with respect to the connector.

6. The delivery device according to claim 5,
wherein the second sensor element is connected to the connector via a keyed connection,
the keyed connection comprising a lug that is slideably received within a longitudinal recess orientated parallel to the longitudinal axis.

7. The delivery device according to claim 1,
wherein the contact elements of the first sensor element are configured as two-dimensional surface contacts.

8. The delivery device according to claim 1,
wherein the contact elements of the first sensor element are arranged on a cylindrical surface that is orientated parallel to the longitudinal axis.

9. The delivery device according to claim 8,
wherein the second sensor element is configured to electrically contact the contact elements of the first sensor element in radial directions perpendicular to the longitudinal axis.

10. The delivery device according to claim 1,
Wherein the dose setting sensor comprises an insulating carrier supporting the electrical conductors and contact elements of the first sensor element.

11. The delivery device according to claim 10,
wherein the insulating carrier is a rigid or free-standing structure,
the electrical conductors or the contact elements of the first sensor element are rigidly attached to the carrier or co-molded with the insulating carrier.

12. The delivery device according to claim 1,
wherein the second sensor element is a conductive metal element with an integrally formed linking structure contacting the first sensor element.

13. The delivery device according to claim 12,
wherein the second sensor element comprises a conductive metal ring holding a plurality of linking elements of the linking structure, and
the linking elements are configured to electrically connect at least two contact structures of the first sensor element with each other.

14. The delivery device according to claim 1,
wherein the dose setting sensor comprises a rotary dose setting encoder configured to generate sensor signals having electrical pulses upon rotation of the controller during dose setting, and
the electronics module is configured to determine a set dose from a number of the electrical pulses generated by the dose setting sensor.

15. The delivery device according to claim 1,
wherein the dose setting sensor is configured to provide a sensor signal to the electronics module indicative of a direction of rotation of the controller.

16. The delivery device according to claim 1,
wherein the electrical conductors comprise a first conductor and a second conductor,
the first sensor elements comprises a first contact structure conductively connected to the first conductor and a second contact structure conductively connected to the second conductor, and
the second sensor element comprises a structure configured to repeatedly open and close an electrical contact between the first and second contact structure upon rotation of the controller.

17. The delivery device according to claim 16,
wherein the linking structure comprises a first linking element and a second linking element conductively connected to the first linking element, and
the second linking element is configured to sequentially move into electrical contact with individual contact elements of the contact elements of the second contact structure upon rotation of the controller while the first linking element is in electrical contact with the first contact structure and to sequentially connect the individual contact elements of the second contact structure with the first contact structure.

18. The delivery device according to claim 17,
wherein the first linking element is configured to conductively contact a single contact element of the contact elements of the first contact structure while the second linking element sequentially moves into the electrical contact with the contact elements of the second contact structure.

19. The delivery device according to claim 17,
wherein the first contact structure and the second contact structure are circumferentially arranged after each other around the longitudinal axis such that,
while a rotational position of the controller is within a first angular range, the first linking element contacts the first contact structure and the second linking element contacts the second contact structure, and
while the rotational position of the controller is within a second angular range, the first linking element contacts the second contact structure, and
while the rotational position of the controller is within a third angular range, the second linking element contacts the first contact structure,
the second angular range different from the third angular range.

20. The delivery device according to claim 16,
wherein the electrical conductors comprise a third conductor,
the first sensor element comprises a third contact structure conductively connected to the third conductor (103, 703),
the linking structure of the second sensor element is configured to repeatedly open and close an electrical contact between the first and third contact structure upon rotation of the controller.

21. The delivery device according to claim 20,
wherein contact elements of the second contact structure and contact elements of the third contact structure are offset with respect to each other so that, upon rotation of the controller, the opening and closing of the electrical contact between the first and second contact structure exhibits a temporal shift with respect to the opening and closing of the electrical contact between the first and third contact structure, and
the electronics module is configured to determine a direction of rotation of the controller from the temporal shift.

22. The delivery device according to claim 4,
wherein the controller is configured to axially move with respect to the housing during dose delivery, and
the sensor mechanism comprises a dose delivery sensor configured to sense a delivery of a set dose by detecting the axial movement of the controller.

23. The delivery device according to claim 22,
wherein the dose delivery sensor comprises a sensor portion that is rotationally fixed with respect to the housing,
the dose delivery sensor comprises an electrical connector configured to conductively connect the rotationally fixed sensor portion to the electronics module,
the electrical connector is configured to be in an open state during dose setting, and
the electrical connector is configured to be transferred into a closed state during delivery of the set dose at a beginning of the delivery of the set dose.

24. The delivery device according to claim 23,
wherein the electrical connector is in the open state when the controller is in the dose setting position,
wherein the electrical connector is transferred into the closed state when the controller is moved into the dose delivery position.

25. The delivery device according to claim 23,
wherein the electrical connector comprises a first part that is rotationally fixed with respect to the controller and the electronics module and that is conductively connected to the electronics module,
the electrical connector comprises a second part that is rotationally fixed with respect to the housing, and
the first part is axially and rotationally movable with respect to the second part.

26. The delivery device according to claim 25,
wherein the electrical connector comprises a circumferential contact arrangement and a connector contact,
the circumferential contact arrangement is circumferentially arranged about the longitudinal axis,
the circumferential contact arrangement is rotationally and axially movable with respect to the connector contact,
the electrical connector is configured to be transferred into the closed state by axial movement of the circumferential contact arrangement with respect to the connector contact, and
the connector contact is configured to electrically contact the circumferential contact arrangement in the closed state of the electrical connector.

27. The delivery device according to claim 26,
wherein individual contacts of the circumferential contact arrangement are circumferentially distributed around the longitudinal axis,
the electrical connector is only transferrable into the closed state if when the circumferential contact arrangement is positioned at distinct and separated rotational positions with respect to the connector contact,
the connector contact is configured to contact different sets of the contacts of the circumferential contact arrangement when being positioned at individual rotational positions of the distinct and separated rotational positions.

28. The delivery device according to claim 26,
wherein the first part comprises the circumferential contact arrangement and the second part comprises the connector contact.

29. The delivery device according to claim 23,
wherein the dose delivery sensor is an end of dose switch configured to be actuated upon full delivery of the set dose.

30. The delivery device according claim 29,
wherein the end of dose switch is actuated by transferring a further electrical connector from an open state into a closed state,
the rotationally fixed sensor portion comprises the further electrical connector, and
the electrical connector is configured to be transferred from the open state to the closed state upon beginning of dose delivery.

31. The delivery device according to claim 22,
wherein the dose delivery sensor is a linear sensor configured to sense axial movement of the controller along the longitudinal axis during dose delivery.

32. The delivery device according to claim 31,
wherein the rotationally fixed sensor portion comprises a dose delivery encoder that is elongated along the longitudinal axis and that is axially movable with respect to the housing.

33. The delivery device according to claim 32,
wherein, upon axial movement, the dose delivery encoder is configured to repeatedly contact a static electrical contact that is axially fixed with respect to the housing, and
the electronics module is configured to monitor the repeated contacting between the dose delivery encoder and the static electrical contact and to determine the axial movement of the controller from the repeated contacting.

34. The delivery device according to claim 31,
wherein the electronics module is configured to determine a set dose from the rotation of the controller sensed by the dose setting sensor,
the electronics module is configured to determine an injected dose from the axial movement sensed by the dose delivery sensor upon a stopping of the axial movement of the controller or upon a release of the controller,
the electronics module is configured to compare the injected dose with the set dose to detect an only partial delivery of the set dose.
